(19) United States
(12) Patent Application Publication
(10) Pub. No.: US 2025/0109166 A1
(43) Pub. Date: Apr. 3, 2025

(54) PROCESS FOR MANUFACTURING MACROCYCLIC PEPTIDES

(71) Applicant: Hoffmann-La Roche Inc., Little Falls, NJ (US)

(72) Inventors: Jean-Michel Adam, Rosenau (FR); Fritz Bliss, Hartheim (DE); Pascal Jean Claude Dott, Rixheim (FR); Fabienne Roxane Hoffmann-Emery, Delémont (CH); Ulf Göran Larsson, Stockholm (SE); Kurt Puentener, Kaiseraugst (CH)

(73) Assignee: Hoffmann-La Roche Inc., Little Falls, NJ (US)

(21) Appl. No.: 18/803,333

(22) Filed: Aug. 13, 2024

Related U.S. Application Data
(63) Continuation of application No. PCT/EP2023/053409, filed on Feb. 13, 2023.

(30) Foreign Application Priority Data
Feb. 14, 2022 (EP) 22156480.0

(51) Int. Cl. C07K 5/09; C07D 209/20; C07D 209/26; C07D 401/12; C07K 1/107

(52) U.S. Cl. CPC C07K 5/0815; C07D 209/20; C07D 209/26; C07D 401/12; C07K 1/1077

(57) ABSTRACT

The invention provides a novel process for manufacturing a compound of formula (I), or a salt thereof,

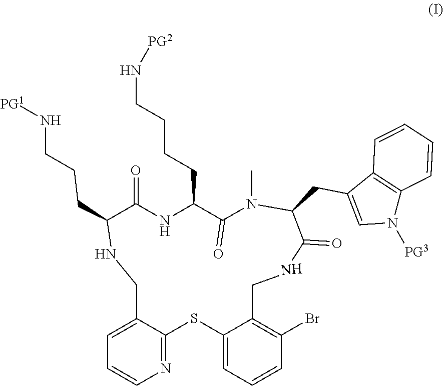

wherein PG¹, PG² and PG³ are amino protective groups. The process according to the invention is particularly suitable for large-scale manufacturing under GMP conditions.

### CROSS-REFERENCE TO RELATED APPLICATIONS

This application is a continuation of International Application No. PCT/EP2023/053409 having an international filing date of Feb. 13, 2023, which claims benefit of priority to European Patent Application No. 22156480.0, filed Feb. 14, 2022, each of which is incorporated herein by reference in its entirety.

### FIELD OF THE INVENTION

The invention relates to a novel process for manufacturing a compound of formula (I), or a salt thereof,

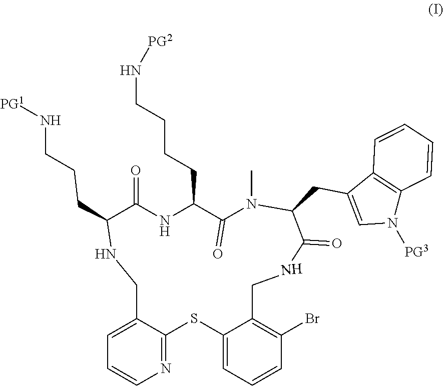

wherein PG¹, PG² and PG³ are amino protective groups. The process according to the invention is particularly suitable for large-scale manufacturing under GMP conditions.

### BACKGROUND OF THE INVENTION

The compound of formula (Ia) is a crucial precursor in the synthesis of the novel antibiotic 1:

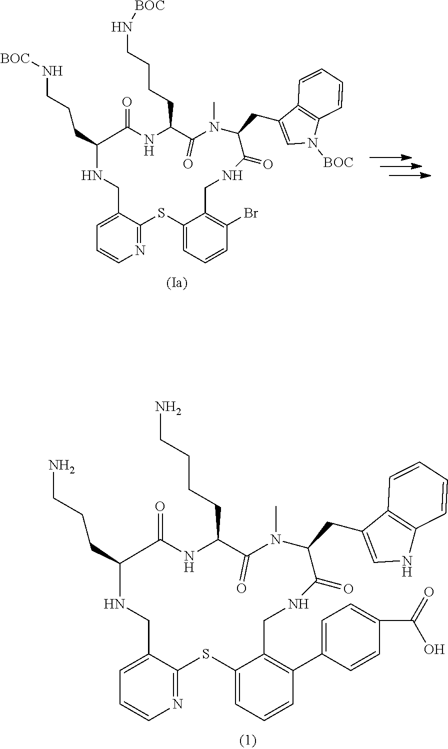 [SMILES: CN1C(=O)[C@H](CCCCNC(=O)OC(C)(C)C)NC(=O)[C@H](CCCNC(=O)OC(C)(C)C)NCc2cccnc2Sc2cccc(Br)c2CNC(=O)[C@@H]1Cc1cn(C(=O)OC(C)(C)C)c2ccccc12 ; CN1C(=O)[C@H](CCCCN)NC(=O)[C@H](CCCN)NCc2cccnc2Sc2cccc(-c3ccc(C(=O)O)cc3)c2CNC(=O)[C@@H]1Cc1c[nH]c2ccccc12]

For marketing products, it is necessary to produce pharmaceuticals in large quantities and according to good manufacturing practice (“GMP”). Hence, high-yielding, cheap, safe and reproducible syntheses are of utmost importance.

WO2019206853 discloses a laboratory scale synthesis of the compound of formula (Ia), which relies on a solid phase synthesis of a particular tripeptide. However, it has been found that said solid-phase synthesis is not suitable for industrial scale manufacturing of the compound of formula (Ia) due to various issues, such as low yields, long reaction times and epimerization of certain stereocentres.

Accordingly, there is a high unmet need for a new process for manufacturing the compound of formula (Ia).

### SUMMARY OF THE INVENTION

The present invention provides a solution phase process for manufacturing compounds of formula (I), which overcomes the problems outlined above. The present invention also provides certain intermediates that are useful in the new process. Finally, the present invention provides a new method for Fmoc deprotection of Fmoc protected amines.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims and the abstract), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims and the abstract), or to any novel one, or any novel combination, of the steps of any method or process so disclosed.

The term “protective group” (PG) denotes a group which selectively blocks a reactive site in a multifunctional compound such that a chemical reaction can be carried out selectively at another unprotected reactive site in the meaning conventionally associated with it in synthetic chemistry. Protective groups can be removed at the appropriate point. Exemplary amino protective groups are Boc (tert-butoxycarbonyl), benzyl, 4-methoxybenzyl, benzhydryl, Fmoc (fluorenylmethoxycarbonyl), Cbz (benzyloxycarbonyl), Moz (p-methoxybenzyloxy carbonyl), Troc (2,2,2-trichloroethoxycarbonyl), Teoc (2-(Trimethylsilyl)ethoxycarbonyl), Adoc (adamantoxycarbonyl), formyl, acetyl, and cyclobutoxycarbonyl. Further particular amino protective groups are tert-butoxycarbonyl (Boc) and fluorenylmethoxycarbonyl (Fmoc).

Exemplary carboxylic acid protective groups are allyl and 9-fluorenylmethyl (Fm). Exemplary amino and carboxylic acid protective groups and their application in organic synthesis are described, for example, in “Protective Groups in Organic Chemistry” by T. W. Greene and P. G. M. Wutts, 5th Ed., 2014, John Wiley & Sons, N.Y, which is included herein by reference in its entirety.

The term “salt” as used herein refers to any kind of salts formed by reacting the compounds disclosed herein with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. Where the compounds disclose herein contain a free acidic moiety, salts may also be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

### Manufacturing Process

In a first aspect, the present invention provides a process for manufacturing a compound of formula (I), or a salt thereof,

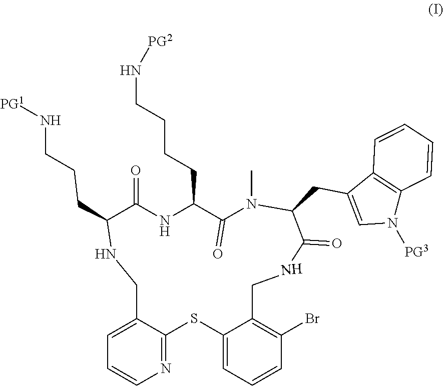

comprising:
(a) reacting a carboxylic acid of formula (II)

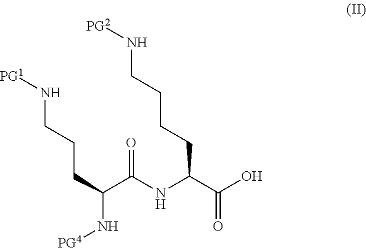

with a secondary amine of formula (III)

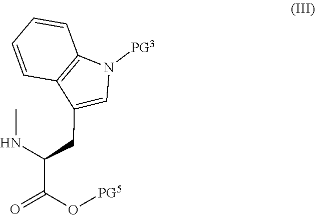

using reagents selected from:
(i) HOAt and DIC;
(ii) HODhat and DIC;
(iii) HOPO and DIC;
(iv) HOPO and DCC; and
(v) HOPO and EDC;
to form a compound of formula (IV)

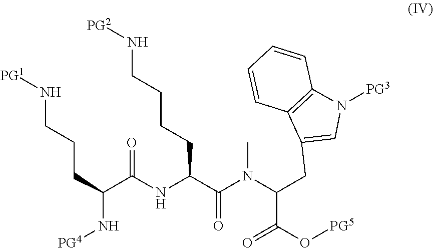

wherein PG¹, PG², PG³ and PG⁴ are amino protective groups, and PG⁵ is a carboxylic acid protective group.

In one embodiment, PG¹, PG², PG³ and PG⁴ are amino protective groups independently selected from BOC, Adoc, Moz, and Fmoc, and PG⁵ is a carboxylic acid protective group selected from allyl and 9-fluorenylmethyl.

It has been found that coupling the carboxylic acid (II) with the secondary amine (III) is very prone to epimerization, as illustrated in comparative example 6. The specific reaction conditions according to the present invention provide a high yield, with very low levels of epimerization.

In a preferred embodiment, the present invention provides a manufacturing process as disclosed herein, wherein the mixture used in step (a) is a mixture of HOAt and DIC.

In a particularly preferred embodiment, the present invention provides a manufacturing process as disclosed herein, wherein the reagents used in step (a) are a mixture of HOPO and DIC.

In one embodiment, the present invention provides a manufacturing process as disclosed herein, wherein step (a) is performed in a solvent selected from:
(i) a mixture of tert-butyl methyl ether, n-heptane and dimethylacetamide;
(ii) isopropylacetate with or without 1,3-dimethyl-2-imidazolidinone;
(iii) tert-butyl methyl ether with or without 1,3-dimethyl-2-imidazolidinone;
(iv) dichloromethane with or without 1,3-dimethyl-2-imidazolidinone;
(v) THE with or without 1,3-dimethyl-2-imidazolidinone;
(vi) 2-methyl-THF with or without 1,3-dimethyl-2-imidazolidinone;
(vii) toluene with or without 1,3-dimethyl-2-imidazolidinone; and
(viii) acetonitrile.

In a preferred embodiment, the present invention provides a manufacturing process as disclosed herein, wherein, in step (a), the reagents used are a mixture of HOAt and DIC and the solvent is a mixture of tert-butyl methyl ether, n-heptane and dimethylacetamide.

In a preferred embodiment, the present invention provides a manufacturing process as disclosed herein, wherein, in step (a), the reagents used are a mixture of HOPO and DIC; and the solvent is selected from:
(i) isopropylacetate with or without 1,3-dimethyl-2-imidazolidinone;
(ii) tert-butyl methyl ether with or without 1,3-dimethyl-2-imidazolidinone;
(iii) dichloromethane with or without 1,3-dimethyl-2-imidazolidinone;
(iv) THE with or without 1,3-dimethyl-2-imidazolidinone;
(v) 2-methyl-THF with or without 1,3-dimethyl-2-imidazolidinone; and
(vi) toluene with or without 1,3-dimethyl-2-imidazolidinone.

In a preferred embodiment, the present invention provides a manufacturing process as disclosed herein, wherein, in step (a), the reagents used are a mixture of HOPO and DIC; and the solvent is selected from:
(i) isopropylacetate with or without 1,3-dimethyl-2-imidazolidinone;
(ii) tert-butyl methyl ether with or without 1,3-dimethyl-2-imidazolidinone;
(iii) 2-methyl-THF with or without 1,3-dimethyl-2-imidazolidinone; and
(iv) toluene with or without 1,3-dimethyl-2-imidazolidinone.

In a particularly preferred embodiment, the present invention provides a manufacturing process as disclosed herein, wherein, in step (a), the reagents used are a mixture of HOPO and DIC; and the solvent is a mixture of isopropylacetate and 1,3-dimethyl-2-imidazolidinone.

In a particularly preferred embodiment, the present invention provides a manufacturing process as disclosed herein, wherein, in step (a), the reagents used are a mixture of HOPO and DIC; and the solvent is a mixture of tert-butyl methyl ether and 1,3-dimethyl-2-imidazolidinone.

In one embodiment, the present invention provides a manufacturing process as disclosed herein, further comprising:
(b1) reacting said compound of formula (IV), wherein PG⁴ is Fmoc, with N-acetylcysteine and tAmNH₂, to form a compound of formula (V):

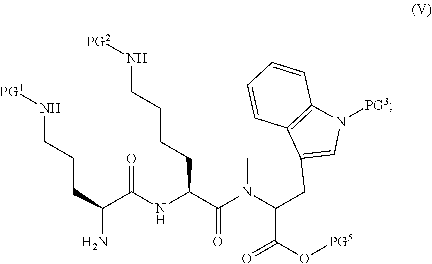

and
(b2) washing the reaction mixture obtained in (b1) with a basic aqueous solution.

In one embodiment, the present invention provides a manufacturing process as disclosed herein, further comprising:
(b1) reacting said compound of formula (IV), wherein PG⁴ is Fmoc, with N-acetylcysteine and tAmNH₂ or tBuNH₂, to form a compound of formula (V):

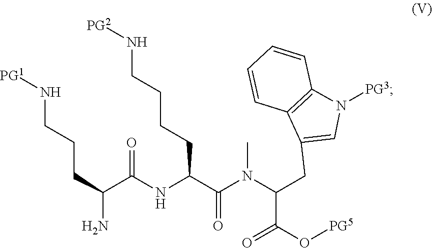

wherein PG¹, PG², and PG³ are amino protective groups independently selected from BOC, Adoc, Moz, and Fmoc, and PG⁵ is a carboxylic acid protective group selected from allyl and 9-fluorenylmethyl; and
(b2) washing the reaction mixture obtained in (b1) with a basic aqueous solution.

In a preferred embodiment, said basic aqueous solution is an aqueous solution of KHCO₃ and/or K₂CO₃.

In a particularly preferred embodiment, said step (b2) consists of:
(b2a) washing the reaction mixture obtained in (b1) with an aqueous solution of KHCO₃; and
(b2b) washing the reaction mixture obtained in (b2a) with an aqueous solution of K₂CO₃.

In one embodiment, the present invention provides a manufacturing process as disclosed herein, further comprising:
(c) reacting said compound of formula (V), with a compound of formula (VI):

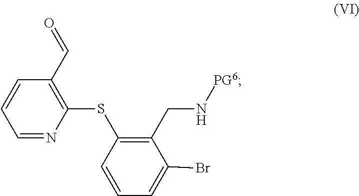

wherein PG⁶ is an amino protective group; in the presence of (i) a reducing agent selected from NaBH₃CN and NaBH(OAc)₃; and (ii) a carboxylic acid selected from acetic acid and propionic acid; to form a compound of formula (VII):

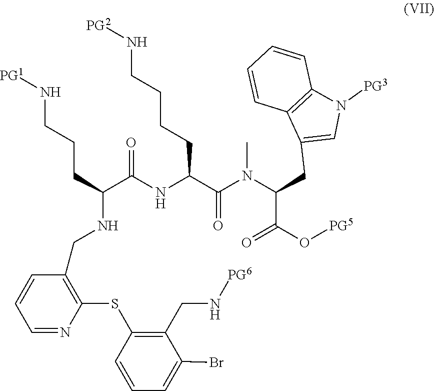

wherein PG¹, PG², PG³ and PG⁶ are amino protective groups, and PG⁵ is a carboxylic acid protective group.

In one embodiment, the present invention provides a manufacturing process as disclosed herein, further comprising:
(c) reacting said compound of formula (V), with a compound of formula (VI):

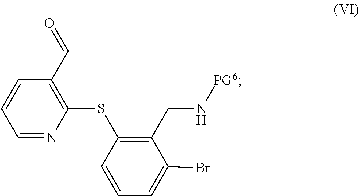

wherein PG⁶ is an amino protective group selected from BOC, Adoc, Moz, and Fmoc; in the presence of (i) a reducing agent selected from NaBH₃CN and NaBH(OAc)₃; and (ii) a carboxylic acid selected from acetic acid and propionic acid; to form a compound of formula (VII):

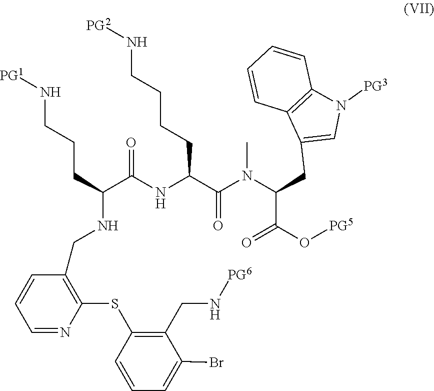

wherein PG¹, PG², PG³ and PG⁶ are amino protective groups independently selected from BOC, Adoc, Moz, and Fmoc, and PG⁵ is a carboxylic acid protective group selected from allyl and 9-fluorenylmethyl.

In one embodiment, the present invention provides a manufacturing process as disclosed herein, wherein, in step (c), said reducing agent is NaBH(OAc)₃ and said carboxylic acid is acetic acid.

In one embodiment, the present invention provides a manufacturing process as disclosed herein, further comprising:
(d) reacting said compound of formula (VII), wherein PG⁵ is an allyl group, with a transition metal catalyst in the presence of a secondary amine, to form a compound of formula (IX):

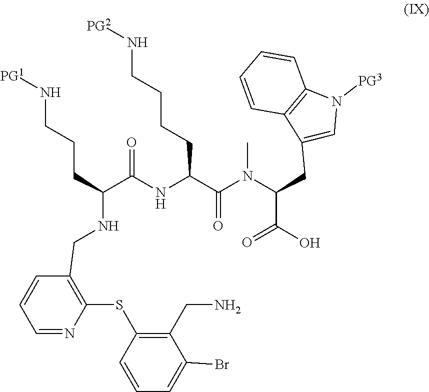

wherein PG¹, PG², PG³ and PG⁶ are amino protective groups.

In one embodiment, the present invention provides a manufacturing process as disclosed herein, further comprising:
(d) reacting said compound of formula (VII), wherein PG⁵ is an allyl group, with a transition metal catalyst in the presence of a secondary amine, to form a compound of formula (IX):

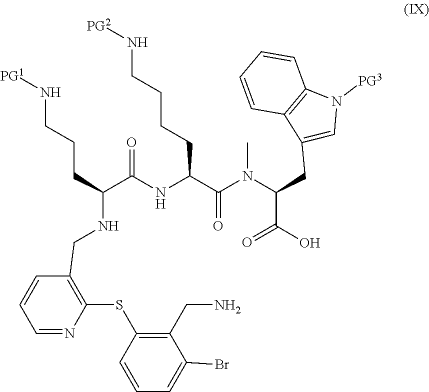

wherein PG¹, PG² and PG³ are amino protective groups independently selected from BOC, Adoc, Moz, and Fmoc.

In one embodiment, said transition metal catalyst used in step (d) is a palladium catalyst.

In a preferred embodiment, said transition metal catalyst used in step (d) is a palladium⁽⁰⁾ catalyst.

In a particularly preferred embodiment, said transition metal catalyst used in step (d) is (PPh₃)₄Pd.

In a particularly preferred embodiment, said secondary amine used in step (d) is Et₂NH.

In a particularly preferred embodiment, said secondary amine used in step (d) is Et₂NH and said transition metal catalyst is (PPh₃)₄Pd.

In one embodiment, the present invention provides a manufacturing process as disclosed herein, wherein step (d) is performed in acetonitrile and further comprises working up the reaction mixture obtained from step (d) by:
(d2) adding N-acetylcysteine to said reaction mixture obtained from step (d);
(d3) adding Cy₂NH to the reaction mixture obtained from step (d2);
(d4) distilling off the secondary amine from step (d); and
(d5) filtering the reaction mixture obtained from (d4).

In one embodiment, the present invention provides a manufacturing process as disclosed herein, further comprising:
(e) reacting said compound of formula (IX) with:
   (i) a mixture of HOBt and EDCI;
   (ii) a mixture of DIC and oxyma;
   (iii) COMU;
   (iv) 2,4,6-trichloro-1,3,5-triazine (TCT); or
   (v) 4-(4,6-dimethoxy-1,3,5-triazin-2-yl)-4-methyl-morpholinium chloride (DMTMM);
      to form a compound of formula (I).

In one embodiment, the present invention provides a manufacturing process as disclosed herein, further comprising:
(e) reacting said compound of formula (IX) with:
   (i) a mixture of HOBt and EDCI; or
   (ii) a mixture of DIC and oxyma; or
   (iii) COMU;
   to form the compound of formula (I).

In one aspect, the present invention provides a process for manufacturing a compound of formula (I), or a salt thereof,

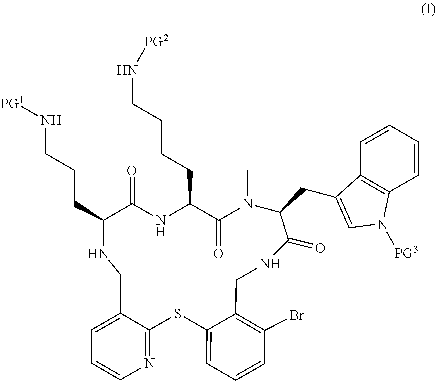

comprising:
(a) reacting a carboxylic acid of formula (IIb)

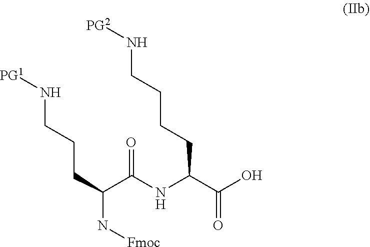

wherein PG¹ and PG² are an amino protective groups, with a secondary amine of formula (IIIb)

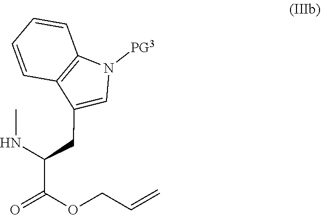

wherein PG³ is an amino protective group, using reagents selected from:
(i) HOAt and DIC; or
(ii) HODhat and DIC; or
(iii) HOPO and DIC; or
(iv) HOPO and DCC; or
(v) HOPO and EDC;
to form a compound of formula (IVb)

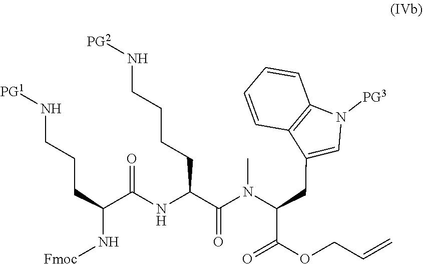

(b1) reacting said compound of formula (IVb), with N-acetylcysteine and tAmNH₂, to form a compound of formula (Vb):

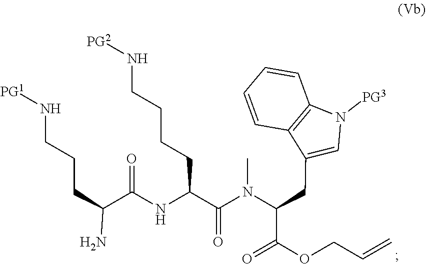

(b2) washing the reaction mixture obtained in (b1) with a basic aqueous solution
(c) reacting said compound of formula (Vb), with a compound of formula (VIa):

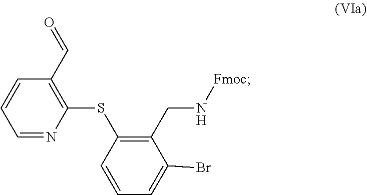

in the presence of (i) a reducing agent selected from NaBH₃CN and NaBH(OAc)₃; and (ii) a carboxylic acid selected from acetic acid and propionic acid; to form a compound of formula (VIIb):

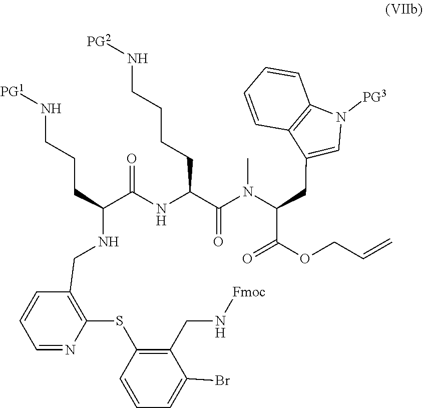

(d) reacting said compound of formula (VIIb) with a transition metal catalyst in the presence of a secondary amine, to form a compound of formula (IX):

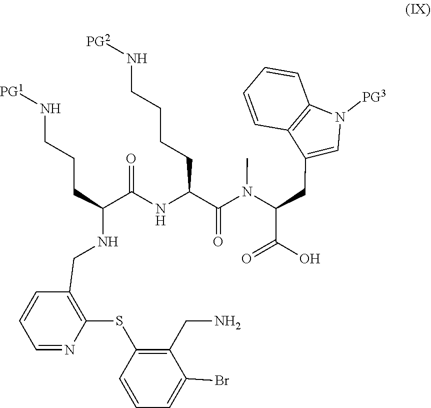

followed by working up the reaction mixture obtained from (d) by:
(d2) adding N-acetylcysteine to said reaction mixture obtained from step (d);
(d3) adding Cy₂NH to the reaction mixture obtained from step (d2);
(d4) distilling off the secondary amine from step (d); and
(d5) filtering the reaction mixture obtained from step (d4); and
(e) reacting said compound of formula (IX) with:
   (i) a mixture of HOBt and EDCI; or
   (ii) a mixture of DIC and oxyma; or
   (iii) COMU;
   to form the compound of formula (I).

In one aspect, the present invention provides a process for manufacturing a compound of formula (IVb) described herein, or a salt thereof, comprising:
(a) reacting a carboxylic acid of formula (IIb) described herein with an amine of formula (IIIb) described herein using reagents selected from:
   (i) HOAt and DIC;
   (ii) HODhat and DIC;
   (iii) HOPO and DIC;
   (iv) HOPO and DCC; and
   (v) HOPO and EDC;
to form said compound of formula (IVb).

In one aspect, the present invention provides a process for manufacturing a compound of formula (Vb) described herein, or a salt thereof, comprising:
(a) reacting a carboxylic acid of formula (IIb) described herein with an amine of formula (IIIb) described herein using reagents selected from:
   (i) HOAt and DIC;
   (ii) HODhat and DIC;
   (iii) HOPO and DIC;
   (iv) HOPO and DCC; and
   (v) HOPO and EDC;
   to form a compound of formula (IVb) described herein;
(b1) reacting said compound of formula (IVb), with N-acetylcysteine and tAmNH₂, to form said compound of formula (Vb); and
(b2) washing the reaction mixture obtained in (b1) with a basic aqueous solution.

In one aspect, the present invention provides a process for manufacturing a compound of formula (VIIb) described herein, or a salt thereof, comprising:
(a) reacting a carboxylic acid of formula (IIb) described herein with an amine of formula (IIIb) described herein using reagents selected from:
   (i) HOAt and DIC;
   (ii) HODhat and DIC;
   (iii) HOPO and DIC;
   (iv) HOPO and DCC; and
   (v) HOPO and EDC;
to form a compound of formula (IVb) described herein;
(b1) reacting said compound of formula (IVb), with N-acetylcysteine and tAmNH₂, to form said compound of formula (Vb);
(b2) washing the reaction mixture obtained in (b1) with a basic aqueous solution; and
(c) reacting said compound of formula (Vb) with a compound of formula (VIa) described herein in the presence of (i) a reducing agent selected from NaBH₃CN and NaBH(OAc)₃; and (ii) a carboxylic acid selected from acetic acid and propionic acid; to form said compound of formula (VIIb).

In one aspect, the present invention provides a process for manufacturing a compound of formula (IX) described herein, or a salt thereof, comprising:
(a) reacting a carboxylic acid of formula (IIb) described herein with an amine of formula (IIIb) described herein using reagents selected from:
   (i) HOAt and DIC;
   (ii) HODhat and DIC;
   (iii) HOPO and DIC;
   (iv) HOPO and DCC; and
   (v) HOPO and EDC;
   to form a compound of formula (IVb) described herein;
(b1) reacting said compound of formula (IVb), with N-acetylcysteine and tAmNH₂, to form said compound of formula (Vb);
(b2) washing the reaction mixture obtained in (b1) with a basic aqueous;
(c) reacting said compound of formula (Vb) with a compound of formula (VIa) described herein in the presence of (i) a reducing agent selected from NaBH₃CN and NaBH(OAc)₃; and (ii) a carboxylic acid selected from acetic acid and propionic acid; to form said compound of formula (VIIb); and
(d) reacting said compound of formula (VIIb) with a transition metal catalyst in the presence of a secondary amine, to form said compound of formula (IX).

In one aspect, the present invention provides a process for manufacturing a compound of formula (IX) described herein, or a salt thereof, comprising:
(a) reacting a carboxylic acid of formula (IIb) described herein with an amine of formula (IIIb) described herein using reagents selected from:
   (i) HOAt and DIC;
   (ii) HODhat and DIC;
   (iii) HOPO and DIC;
   (iv) HOPO and DCC; and
   (v) HOPO and EDC;
   to form a compound of formula (IVb) described herein;
(b1) reacting said compound of formula (IVb), with N-acetylcysteine and tAmNH₂, to form said compound of formula (Vb);
(b2) washing the reaction mixture obtained in (b1) with a basic aqueous;
(c) reacting said compound of formula (Vb) with a compound of formula (VIa) described herein in the presence of (i) a reducing agent selected from NaBH₃CN and NaBH(OAc)₃; and (ii) a carboxylic acid selected from acetic acid and propionic acid; to form said compound of formula (VIIb); and
(d) reacting said compound of formula (VIIb) with a transition metal catalyst in the presence of a secondary amine, to form said compound of formula (IX); followed by working up the reaction mixture obtained from step (d) by:
   (d2) adding N-acetylcysteine to said reaction mixture obtained from step (d);
   (d3) adding Cy₂NH to the reaction mixture obtained from step (d2);
   (d4) distilling off the secondary amine from step (d); and
   (d5) filtering the reaction mixture obtained from step (d4).

In a particularly preferred embodiment, the present invention provides a process for manufacturing a compound of formula (Ia), which is:

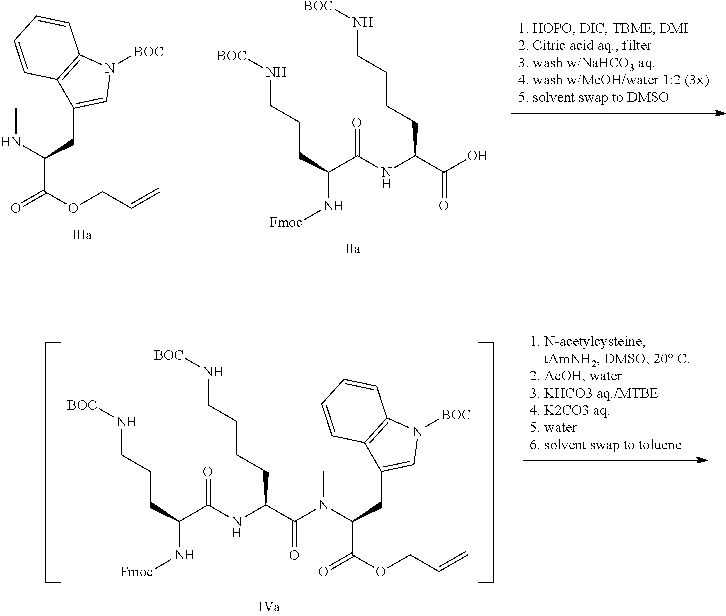

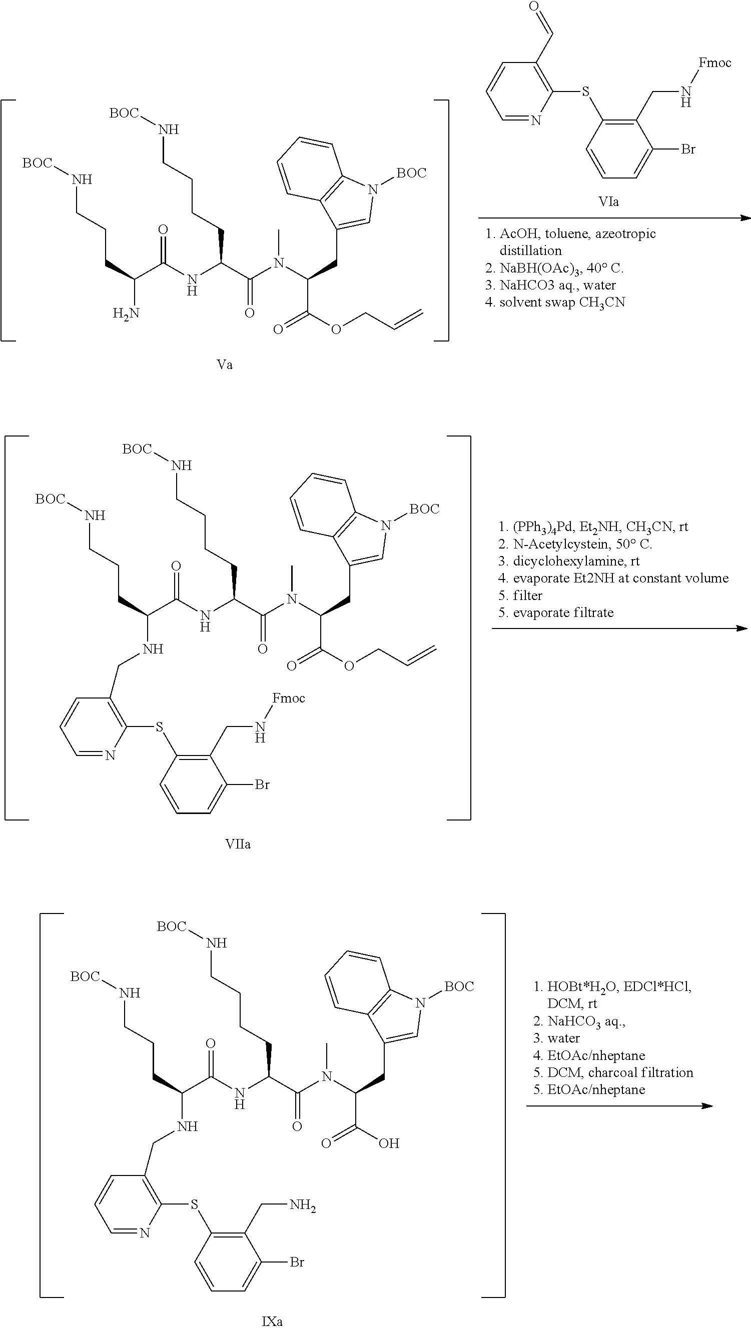

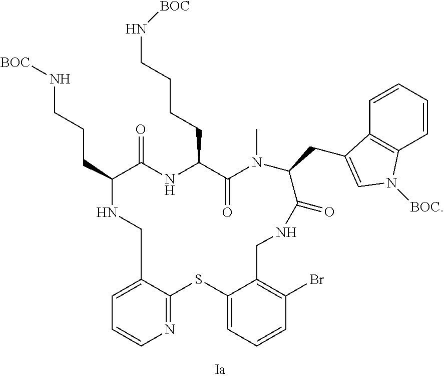 [SMILES: CN1C(=O)[C@H](CCCCNC(=O)OC(C)(C)C)NC(=O)[C@H](CCCNC(=O)OC(C)(C)C)NCc2cccnc2Sc2cccc(Br)c2CNC(=O)[C@@H]1Cc1cn(C(=O)OC(C)(C)C)c2ccccc12]

In one aspect, the present invention provides a compound of formula (I) described herein, or a salt thereof,

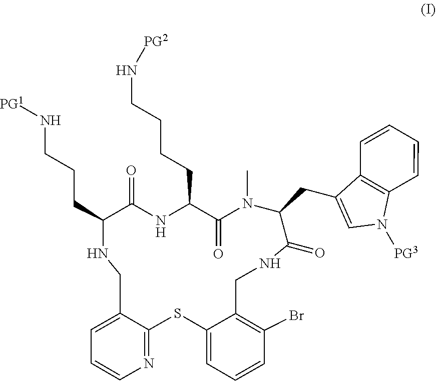

when manufactured according to the processes of the invention.

In one aspect, the present invention provides a process for manufacturing a compound of formula (1), or a salt thereof,

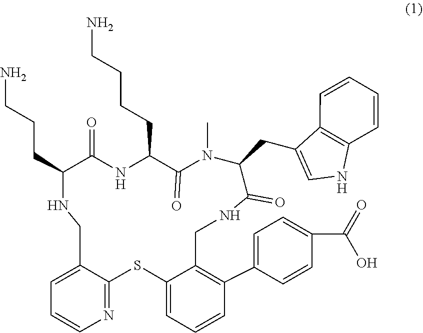 [SMILES: CN1C(=O)[C@H](CCCCN)NC(=O)[C@H](CCCN)NCc2cccnc2Sc2cccc(-c3ccc(C(=O)O)cc3)c2CNC(=O)[C@@H]1Cc1c[nH]c2ccccc12]

comprising any of the processes described herein.

In one aspect, the present invention provides the use of any of the process described herein in the manufacture of the compound of formula (1)

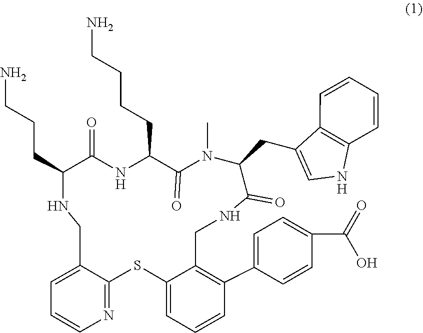 [SMILES: CN1C(=O)[C@H](CCCCN)NC(=O)[C@H](CCCN)NCc2cccnc2Sc2cccc(-c3ccc(C(=O)O)cc3)c2CNC(=O)[C@@H]1Cc1c[nH]c2ccccc12]

or a salt thereof.

In one embodiment, in the processes and compounds disclosed herein, PG¹ is BOC.

In one embodiment, in the processes and compounds disclosed herein, PG² is BOC.

In one embodiment, in the processes and compounds disclosed herein, PG³ is BOC.

In one embodiment, in the processes and compounds disclosed herein, PG⁴ is Fmoc.

In one embodiment, in the processes and compounds disclosed herein, PG⁵ is allyl.

In one embodiment, in the processes and compounds disclosed herein, PG⁶ is Fmoc.

In one embodiment, in the processes and compounds disclosed herein:
PG¹ is BOC;
PG² is BOC; and
PG⁴ is Fmoc.

In one embodiment, in the processes and compounds disclosed herein:
PG³ is BOC; and
PG⁵ is allyl.

In one embodiment, in the processes and compounds disclosed herein:
PG¹ is BOC;
PG² is BOC;
PG³ is BOC;
PG⁴ is Fmoc; and
PG⁵ is allyl.

In one embodiment, in the processes and compounds disclosed herein:
PG¹ is BOC;
PG² is BOC;
PG³ is BOC; and
PG⁵ is allyl.

In one embodiment, in the processes and compounds disclosed herein:
PG¹ is BOC;
PG² is BOC;
PG³ is BOC;
PG⁵ is allyl; and
PG⁶ is Fmoc.

In one embodiment, in the processes and compounds disclosed herein:
PG¹ is BOC;
PG² is BOC; and
PG³ is BOC.

In one aspect, the present invention provides a compound of formula (II)

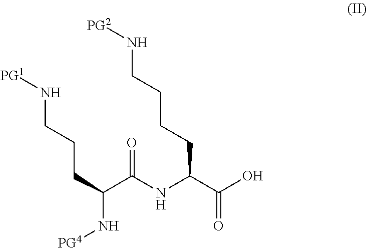

or a salt thereof, wherein PG¹, PG² and PG⁴ are amino protective groups.

In one embodiment, the present invention provides a compound of formula (II) as described herein, or a salt thereof, wherein PG¹, PG² and PG⁴ are amino protective groups independently selected from BOC, Adoc, Moz, and Fmoc.

In a preferred embodiment, the present invention provides a compound of formula (II) as described herein, or a salt thereof, wherein said compound of formula (II) is a compound of formula (IIa)

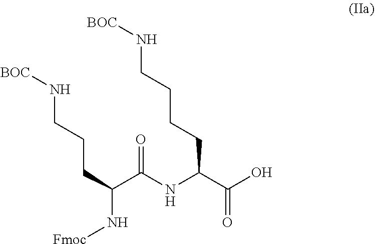

In one aspect, the present invention provides a compound of formula (III)

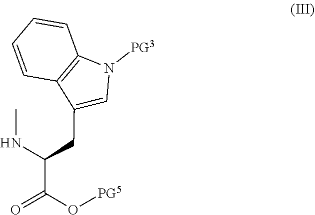

or a salt thereof, wherein PG³ is an amino protective group and PG⁵ is a carboxylic acid protective group.

In one embodiment, the present invention provides a compound of formula (III) as described herein, or a salt thereof, wherein PG³ is an amino protective group selected from BOC, Adoc, Moz, and Fmoc, and PG⁵ is a carboxylic acid protective group selected from allyl and 9-fluorenylmethyl.

In a preferred embodiment, the present invention provides a compound of formula (III) as described herein, or a salt thereof, wherein said compound of formula (III) is a compound of formula (IIIa)

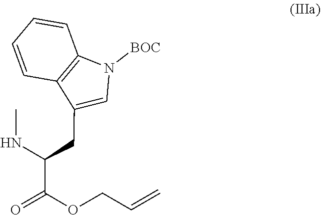 [SMILES: C=CCOC(=O)[C@H](Cc1cn(C(=O)OC(C)(C)C)c2ccccc12)NC]

In one aspect, the present invention provides a compound of formula (IV)

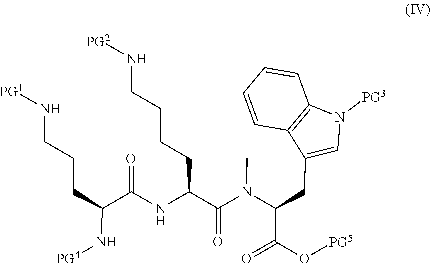

or a salt thereof, wherein PG¹, PG², PG³ and PG⁴ are amino protective groups, and PG⁵ is a carboxylic acid protective group.

In one embodiment, the present invention provides a compound of formula (IV) as described herein, or a salt thereof, wherein PG¹, PG², PG³ and PG⁴ are amino protective groups independently selected from BOC, Adoc, Moz, and Fmoc, and PG⁵ is a carboxylic acid protective group selected from allyl and 9-fluorenylmethyl.

In a preferred embodiment, the present invention provides a compound of formula (IV) as described herein, or a salt thereof, wherein said compound of formula (IV) is a compound of formula (IVa)

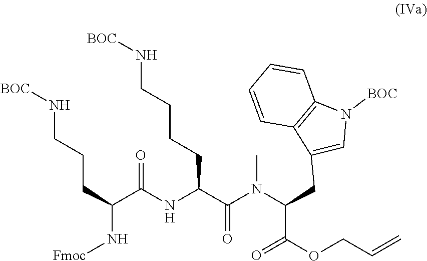

In one aspect, the present invention provides a compound of formula (V)

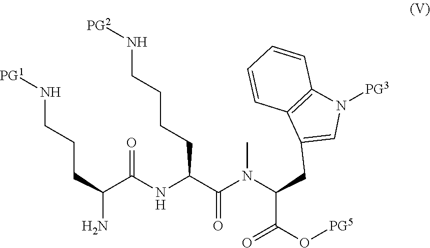

or a salt thereof, wherein PG¹, PG² and PG³ are amino protective groups, and PG⁵ is a carboxylic acid protective group.

In one embodiment, the present invention provides a compound of formula (V) as described herein, or a salt thereof, wherein PG¹, PG² and PG³ are amino protective groups independently selected from BOC, Adoc, Moz, and Fmoc, and PG⁵ is a carboxylic acid protective group selected from allyl and 9-fluorenylmethyl.

In a preferred embodiment, the present invention provides a hemiphosphate salt of the compound of formula (V) as described herein.

In a preferred embodiment, the present invention provides a compound of formula (V) as described herein, or a salt thereof, wherein said compound of formula (V) is a compound of formula (Va)

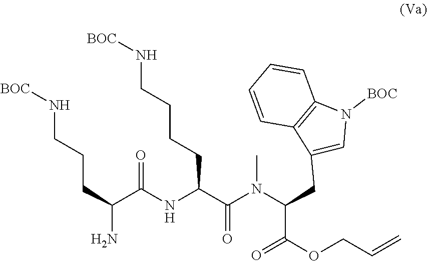 [SMILES: C=CCOC(=O)[C@H](Cc1cn(C(=O)OC(C)(C)C)c2ccccc12)N(C)C(=O)[C@H](CCCCNC(=O)OC(C)(C)C)NC(=O)[C@@H](N)CCCNC(=O)OC(C)(C)C]

In a preferred embodiment, the present invention provides a hemiphosphate salt of the compound of formula (Va) as described herein.

In one aspect, the present invention provides a compound of formula (VI)

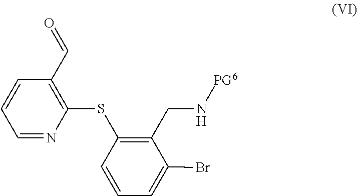

as described herein, or a salt thereof.

In one embodiment, the present invention provides a compound of formula (VI) as described herein, or a salt thereof, wherein PG⁶ is an amino protective group selected from BOC, Adoc, Moz, and Fmoc.

In a preferred embodiment, the present invention provides a compound of formula (VI) as described herein, or a salt thereof, wherein said compound of formula (VI) is a compound of formula (VIa)

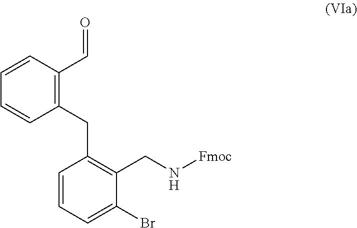

In one aspect, the present invention provides a compound of formula (VII)

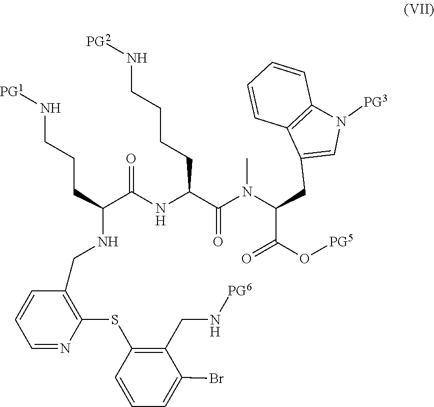

or a salt thereof, wherein PG¹, PG², PG³ and PG⁶ are amino protective groups, and PG⁵ is a carboxylic acid protective group.

In one embodiment, the present invention provides a compound of formula (VII) as described herein, or a salt thereof, wherein PG¹, PG², PG³ and PG⁶ are amino protective groups independently selected from BOC, Adoc, Moz, and Fmoc, and PG⁵ is a carboxylic acid protective group selected from allyl and 9-fluorenylmethyl.

In a preferred embodiment, the present invention provides a compound of formula (VII) as described herein, or a salt thereof, wherein said compound of formula (VII) is a compound of formula (VIIa)

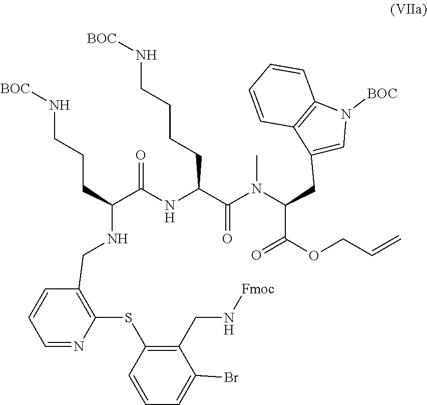

In one aspect, the present invention provides a compound of formula (VIII)

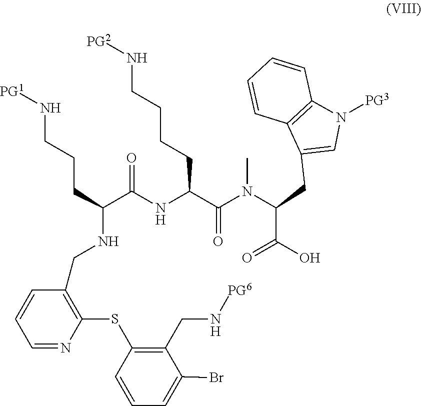

or a salt thereof, PG¹, PG² and PG³ and PG⁶ are amino protective groups. In one embodiment, the present invention provides a compound of formula (VIII) as described herein, or a salt thereof, wherein PG¹, PG² and PG³ and PG⁶ are amino protective groups independently selected from BOC, Adoc, Moz, and Fmoc.

In a preferred embodiment, the present invention provides a compound of formula (VIII) as described herein, or a salt thereof, wherein said compound of formula (VIII) is a compound of formula (VIIIa)

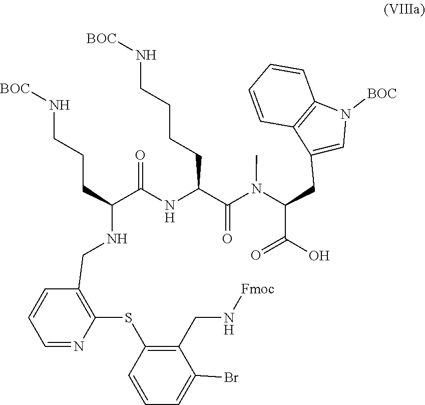

In one aspect, the present invention provides a compound of formula (IX)

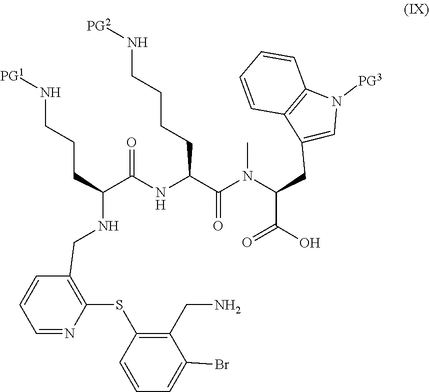

or a salt thereof, wherein PG¹, PG² and PG³ are amino protective groups.

In one embodiment, the present invention provides a compound of formula (IX) as described herein, or a salt thereof, wherein PG¹, PG² and PG³ are amino protective groups independently selected from BOC, Adoc, Moz, and Fmoc.

In a preferred embodiment, the present invention provides a compound of formula (IX) as described herein, or a salt thereof, wherein said compound of formula (IX) is a compound of formula (IXa)

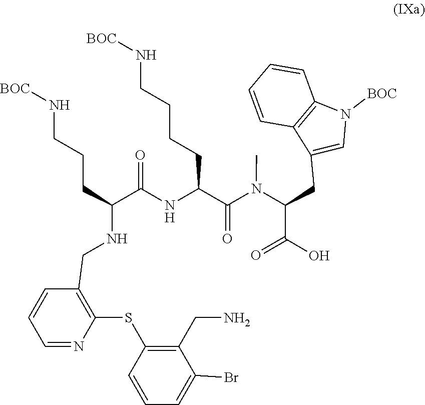 [SMILES: CN(C(=O)[C@H](CCCCNC(=O)OC(C)(C)C)NC(=O)[C@H](CCCNC(=O)OC(C)(C)C)NCc1cccnc1Sc1cccc(Br)c1CN)[C@@H](Cc1cn(C(=O)OC(C)(C)C)c2ccccc12)C(=O)O]

### Novel Method for Fmoc Deprotection

U.S. Pat. No. 9,334,302 discloses a method of removing a Fmoc protective group from an amine, comprising the addition of a mercaptocarboxylic acid to the dibenzofulvene (DBF) Fmoc deprotection byproduct, followed by the removal of the adduct by a basic aqueous wash:

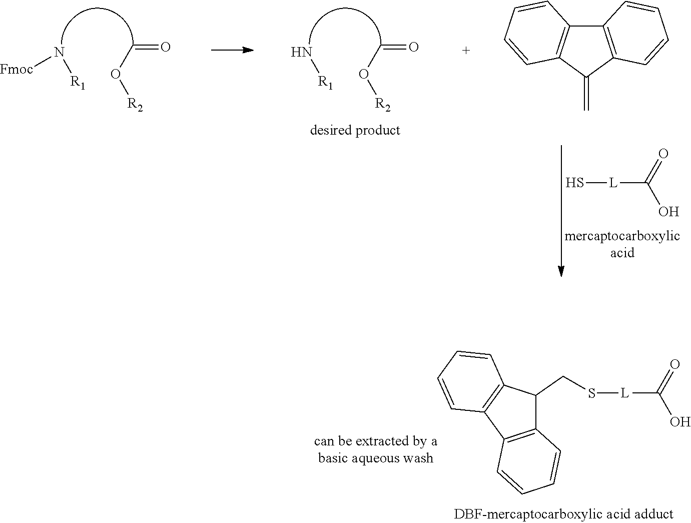

This process may not be applicable if, for example, the desired product contains an acidic functional group (R2=H), since it would be brought into the aqueous phase together with the DBF-mercaptocarboxylic acid adduct. Also, performing a basic aqueous extraction may be inconvenient when performed on an industrial scale.

The present invention therefore provides a new method for Fmoc deprotection, whereby said byproduct is removed by simple filtration, rather than washing with a basic aqueous solution. It has been surprisingly found that t-amylamine and dicyclohexylamine can form salts with the DBF-N-acetylcysteine adduct and that those salts exhibit a poor solubility in selected organic solvents, like MeCN:

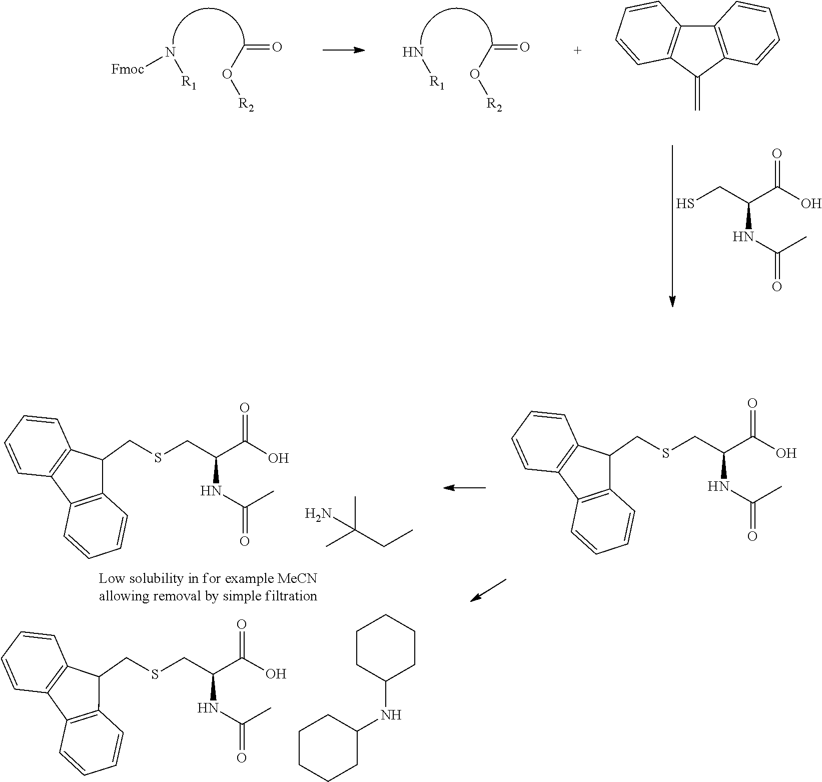

In more detail, the Fmoc protective group is removed by reacting a Fmoc protected amine with a base, such as diethylamine, dicyclohexylamine or t-amylamine. The dibenzofulvene byproduct that is formed in this reaction, is then captured by forming an adduct with N-acetylcysteine. If the base that was used for the removal of the Fmoc protective group was a base other than dicyclohexylamine or t-amylamine, the adduct is subsequently reacted with dicyclohexylamine or t-amylamine. This results in the formation an insoluble salt, which precipitates from the reaction mixture and can conveniently be filtered off.

If the base used to effect the Fmoc deprotection is an amine other than dicyclohexylamine or t-amylamine, e.g., diethylamine, it may also form a salt with the N-acetylcysteine DBF adduct which does not precipitate from the reaction mixture. The presence of such a base may therefore hamper the desired removal of the adduct salt by filtration. It may hence be desirable to remove an excess of this amine reagent by distillation, driving the equilibrium towards the adduct dicyclohexylamine or t-amylamine salt, hence maximizing the precipitation of the corresponding dicyclohexylamine or t-amylamine salt.

Examples 10, 11 and 13 demonstrate the applicability and efficiency of the new method for Fmoc deprotection according to the present invention. The method is further illustrated by the following reaction scheme:

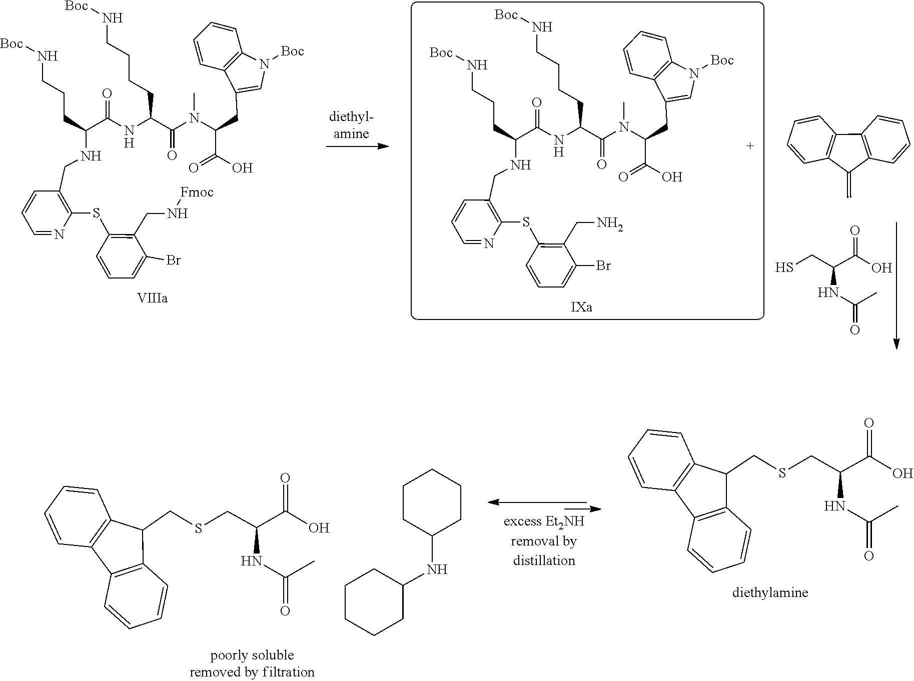

Indeed, in this particular case, the product IXa comprises a carboxylic acid functional group which would preclude a simple basic aqueous wash to remove any DBF-mercaptocarboxylic acid adduct side products.

In one aspect, the present invention provides a method for removing an Fmoc protective group from a compound comprising an Fmoc protected amine, comprising:
(a) reacting said compound comprising an Fmoc protected amine with a base;
(b) adding N-acetylcysteine to the reaction mixture obtained from step (a);
(c) provided the base in step (a) was not Cy₂NH or t-amylamine, adding a base selected from Cy₂NH and t-amylamine to the reaction mixture obtained from step (b);
(d) optionally distilling off the base from step (a); and
(e) filtering the reaction mixture obtained from step (b), (c) or (d).

It is to be understood that steps (a) and (b) do not need to be performed sequentially, but may be performed simultaneously, i.e., in a “one-pot” fashion. Thus, in one embodiment, the present invention provides a method for removing a Fmoc protective group, comprising:
(a) reacting a compound comprising an Fmoc protected amine with a reagent mixture comprising a base and N-acetylcysteine;
(b) provided the base in step (a) was not Cy₂NH or t-amylamine, adding a base selected from Cy₂NH and t-amylamine to the reaction mixture obtained from step (a);
(c) optionally distilling off the base from step (a); and
(d) filtering the reaction mixture obtained from step (a), (b) or (c).

In a preferred embodiment, the method is performed in acetonitrile as a solvent.

In a preferred embodiment, the base used in step (a) is diethylamine.

In a preferred embodiment, the base used in step (c) is Cy₂NH.

In a preferred embodiment, the method for removing a Fmoc protective group according to the invention comprises step (d), distilling off the base from step (a).

In one embodiment, the reaction mixture obtained in step (b) is heated to 30° C. to reflux.

In one embodiment, the reaction mixture obtained in step (b) is heated to 30° C. to 70° C.

In a preferred embodiment, the reaction mixture obtained in step (b) is heated to 40° C. to 60° C.

In a particularly preferred embodiment, the reaction mixture obtained in step (b) is heated to 50° C.

It has surprisingly been found that if ≥10 equivalents of base are used in step (a) of the method according to the invention, the Fmoc deprotection proceeds smoothly at room temperature. Thus, in a preferred embodiment, ≥10 equivalents of base are used relative to compound comprising an Fmoc protected amine in step (a) of the method according to the invention. In a further preferred embodiment, steps (a)-(c) of the method according to the invention are performed at room temperature. In a further preferred embodiment, ≥10 equivalents of base are used relative to the compound comprising an Fmoc protected amine in step (a) of the method according to the invention, and steps (a)-(c) of the method according to the invention are performed at room temperature.

In a preferred embodiment, the method for removing a Fmoc protective group according to the invention comprises:
(a) reacting a compound comprising an Fmoc protected amine with Et₂NH in acetonitrile;
(b) adding N-acetylcysteine to the reaction mixture obtained from (a);
(c) adding Cy₂NH to the reaction mixture obtained from (b);
(d) distilling off Et₂NH; and
(e) filtering the reaction mixture obtained from (d).

In a preferred embodiment, the method for removing a Fmoc protective group according to the invention comprises:
(a) reacting a compound comprising an Fmoc protected amine with a base selected from Cy₂NH and t-amylamine in acetonitrile;
(b) adding N-acetylcysteine to the reaction mixture obtained from (a); and
(c) filtering the reaction mixture obtained from (b).

In a preferred embodiment, the method for removing a Fmoc protective group according to the invention comprises:
(a) reacting a compound comprising an Fmoc protected amine with a reagent mixture comprising N-acetylcysteine and a base selected from Cy₂NH and t-amylamine in acetonitrile; and
(b) filtering the reaction mixture obtained from (a).

In one embodiment, the reagent mixture used in step (a) further comprises seed crystals of a DBF-N-acetylcysteine adduct Cy₂NH or t-amylamine salt.

As mentioned above, the basic aqueous wash described in U.S. Pat. No. 9,334,302 may not be applicable if, for example, the desired product contains an acidic functional group, since it would be washed out into the aqueous phase together with the DBF-mercaptocarboxylic acid adduct byproduct. Thus, in a preferred embodiment, the compound comprising an Fmoc protected amine used in the method according to the invention further comprises at least one carboxylic acid moiety, preferably one to three carboxylic acid moieties, most preferably one carboxylic acid moiety.

### EXAMPLES

The invention will be more fully understood by reference to the following examples. The claims should not, however, be construed as limited to the scope of the examples.

The following abbreviations are used in the present text:

All=allyl; Boc=tert-butoxycarbonyl; BOP-Cl=Bis(2-oxo-3-oxazolidinyl)phosphinic chloride; COMU=(1-cyano-2-ethoxy-2-oxoethylideneaminooxy)dimethylamino-morpholine-carbenium hexafluorophosphate; Cy₂NH: dicyclohexylamine; DBF=dibenzofulvene; DCM=dichloromethane; DIPEA=diisopropylethylamine; DIC=N,N′-diisopropylcarbodiimide; DMAP=4-dimethylaminopyridine; DMF=N,N-dimethylformamide; DMSO=dimethylsulfoxide; DMTMM=4-(4,6-dimethoxy-1,3,5-triazin-2-yl)-4-methylmorpholinium chloride; EDCI=1-(3-dimethylaminopropyl)-3-ethylcarbodiimide hydrochloride; Fmoc=fluorenylmethoxycarbonyl; HATU=2-(7-aza-1H-benzotriazole-1-yl)-1,1,3,3-tetramethyluronium hexafluorophosphate; HOAt=1-hydroxy-7-azabenzotriazole; HOBt=1-Hydroxybenzotriazole; HODhat=3-hydroxypyrido[3,2-d][1,2,3]triazin-4(3H)-one; HOPO=2-hydroxypyridine-N-oxide; IPC=in process control; KFT=Karl Fischer titration; NMM=N-methylmorpholine; Oxyma=ethyl cyanohydroxyiminoacetate; PyBrop=Bromo-tris-pyrrolidino-phosphonium hexafluorophosphate; Su=succinimide; T=temperature; T3P=propylphosphonic anhydride; TBTU=N,N,N′,N′-tetramethyl-O-(benzotriazol-1-yl)uranium tetrafluoroborate; THF=tetrahydrofurane; TOTT=S-(1-Oxido-2-pyridyl)-N,N,N′,N′-tetramethylthiuronium tetrafluoroborate.

### Analytical Methods

HPLC method A for separation of (IVa) and its epimers

| | |
|---|---|
| Stationary phase | Silica with a chiral selector, 3 μm (e.g. |
| | Chiral Technologies CHIRALPAK ® IE-3) |
| Mobile phase | 5.0% v/v water in MeOH |
| Gradient | isocratic |
| Detection | UV, 205 nm |

HPLC method B for separation of (Va) and its epimers

| | |
|---|---|
| Stationary phase | Silica with a chiral selector, 3 μm (e.g. |
| | Chiral Technologies CHIRALPAK ® IE-3) |
| Mobile phase A | Heptane |
| Mobile phase B | Ethanol + 0.2% v/v Diethylamine |
| Gradient | 80% mobile phase A, 20% mobile phase |
| | B, isocratic |
| Detection | UV, 230 nm |

HPLC method C for separation of (Ia) and its epimers

| | |
|---|---|
| Stationary phase | Silica with a chiral selector, 3 μm (e.g. |
| | Chiral Technologies CHIRALPAK ® IC-3) |
| Mobile phase A | Heptane |
| Mobile phase B | Ethanol |
| Gradient | 85% mobile phase A, 15% mobile phase |
| | B, isocratic |
| Detection | UV, 230 nm |

IPC method of comparative Example 6

| | |
|---|---|
| Stationary phase | Symmetry Shield RP8 4.6 × 50 mm |
| Mobile phase A | water/MeCN 95:5, 0.1% vol/vol formic |
| | acid |
| Mobile phase B | MeCN/water 95:5 |
| Gradient | 0-100% B in 8.6 min |
| Detection | UV, 265 nm |

Epimer detection method of comparative Example 6

| | |
|---|---|
| Stationary phase | Zorbax RX-Sil, 250 × 4.6 mm |
| Mobile phase A | n-Heptane |
| Mobile phase B | 2-propanol |
| Gradient | 95-93% A in 3 min, hold for 7 min, 93-40% |
| | A in 0.5 min, hold for 4.5 min |
| Detection | UV, 264 nm |

### Example 1

### Preparation of Fmoc-Orn(Boc)-Lys(Boc)-OH (IIa)

### Step 1: Fmoc-Orn(Boc)-OSu

Fmoc-Orn(Boc)-OH (50.0 g, 0.11 mol), N-hydroxysuccinimide (13.9 g, 0.12 mol) and THE (200 mL) were charged into a reaction vessel. A solution of dicyclohexylcarbodiimide (26.1 g, 0.13 mol) in THE (50 mL) was added within 2 hours at 20° C. The reaction mixture was stirred for further 21 hours. The precipitate was filtered off and washed with THE (30 mL) twice. The filtrate was concentrated at 40-45° C. to 125 mL. EtOAc (150 mL) was added and the mixture was concentrated at 40-45° C. to 165 mL. Isopropyl alcohol (500 mL) was added at room temperature within 1 hour. The suspension was stirred at room temperature for 10 hours. The solid was filtered off, washed with isopropylalcohol (50 mL) twice and dried at 40° C. for 8 hours to afford Fmoc-Orn(Boc)-OSu as a white solid. Typically, a yield of 85% was obtained.

### Step 2: Fmoc-Orn(Boc)-Lys(Boc)-OH (IIa)

H-Lys(Boc)-OH (66.0 g, 0.27 mol) and THE (960 mL) were charged into a reaction vessel. Bis(trimethylsilyl)acetamide (79.3 g, 0.39 mol) was added at 20° C. over 1 hour. The mixture was stirred at 40-45° C. for 2 hours (solid should be completely dissolved) and cooled to 20-25° C. (=solution A). In another reaction vessel, Fmoc-Orn(Boc)-OSu (120 g, 0.22 mol) was dissolved in THE (480 mL) (about 30 minutes) and the solution cooled to 14-16° C. Solution A was added within 1-2 hours keeping the internal temperature at max. 15° C. The reaction mixture was further stirred for 5 hours. The reaction mixture was added to water (1200 mL) cooled to 5-10° C. over 2 hours and the mixture was further stirred at 5-10° C. for 30 minutes. Ethyl acetate (600 mL) was added. The pH was adjusted to 2-3 by addition of 15% aqueous HCl (10-11 g) keeping the temperature at 5-10° C. The phases were separated. The organic layer was washed with aqueous NaCl solution 1.6% (600 g) five times. Ethyl acetate (600 mL) was added and the mixture was concentrated to 780-820 g. This operation was repeated four times with the addition of ethyl acetate (480 mL). After the last addition, the mixture was concentrated to 960-1000 g. The suspension was heated to 40° C. nHeptane (1500 mL) was added over 1 hour. The suspension was further stirred for 1 hour, cooled to 25° C. and stirred 3 hours at this temperature. The precipitate was filtered off, washed with nheptane (300 mL) three times and dried at 45° C. to afford IIa as a white solid. Typically, the yield was about 90%.

LCMS: 705.35 (M+Na⁺) Example 2

### Preparation of N-Me-Trp(Boc)-OAll Oxalate (IIIa)

### Step 1: N-NBS-Trp-OAll

H-N-Trp-OAll hydrochloride (105.0 g, 374.0 mmol), 2-nitrobenzene sulfonylchloride (83.7 g, 377.8 mmol) and ethyl acetate (1050 mL) were charged into a reaction vessel and the mixture cooled to 0-5° C. Water (315 mL) was charged over 30 minutes. Then aqueous KOH 15% (338.0 g) was charged over 4 hours keeping the temperature below 5° C. and the pH=8-9. The reaction mixture was stirred for another 5 hours at 0-5° C. and the pH was adjusted to 5-6 with aqueous HCl 15%. The mixture was brought to room temperature and the phases were separated. The organic layer was washed with water (260 mL). The biphasic mixture was polish filtered over celite. The phases were separated and the organic phase was concentrated under reduced pressure to about 300 g. Ethyl acetate (540 mL) was added and the solution was concentrated under reduced pressure to about 300 g. A Karl Fischer titration was performed and water level should ≤0.05% w/w. Typically the yield was around 98%.

### Step 2: N-NBS-N-Me-Trp-OAll

A 20% solution of N-NBS-Trp-OAll (600 g, 279.4 mmol), potassium carbonate (57.9 g, 418.9 mmol) and tetrabutylammonium bromide (9.01 g, 27.1 mmol) were charged into a reaction vessel. Methyl iodide (47.6 g, 335.4 mmol) was added at room temperature within 30 minutes. The reaction mixture was stirred at 30-35° C. for 15 hours, then cooled to 0-5° C. Water (480 mL) was added over 1 hour and the bisphasic mixture was stirred for 30 minutes. The phases were separated and the organic layer was washed with aqueous NaCl 5% (240 mL) twice. The organic layer was concentrated under reduced pressure to 200-230 g. Ethyl acetate (300 mL) was added and the solution was concentrated to 200-230 g. This operation was repeated once more. A Karl Fischer titration was performed and water level should be 0.05% w/w. A microfiltration was performed to remove the salts. Typically the yield was around 97-98% and the assay 22.5-23.0% w/w.

### Step 3: N-NBS-N-Me-Trp(Boc)-OAll

A 22.8% w/w of N-NBS-N-Me-Trp-OAll in ethyl acetate (529 g, 272.0 mmol), ethyl acetate (200 mL) and DMAP (6.65 g, 54.4 mmol) were charged into a reaction vessel. The reaction mixture was heated to 27° C. and di-tert-butyl dicarbonate (71.2 g, 326.2 mmol) dissolved in ethyl acetate (64.5 mL) was added over 1.5 hours. The reaction mixture was stirred for another 2 hours at 27° C., then heated to 70° C. and cooled to 30° C. over 2 hours. The reaction mixture was concentrated under reduced pressure at 40-45° C. to about 305 g and cooled to room temperature. Methanol (845 mL) was charged within 1 hour and the mixture was stirred for 3 hours. The precipitate was filtered off, washed with methanol (100 mL) three times and dried under reduced pressure at 45° C. for 6 hours to give 133 g (90%) of N-NBS-N-Me-Trp(Boc)-OAll as a light yellow oil. Purification: The yellow oil obtained was dissolved in ethyl acetate (789 mL) at 70° C. and cooled to 30° C. within 2 hours. The mixture was concentrated under reduced pressure at 40° C. to about 277 g and the suspension obtained was cooled to 20-25° C. Methanol (931 mL) was added within 1 hour and the suspension further stirred for 3 hours. The precipitate was filtered off, washed with methanol (67 mL) three times and dried under reduced pressure at 45° C. for 6 hours to afford 127.7 g (96%) of N-NBS-N-Me-Trp(Boc)-OAll as a light yellow solid.

### Step 4: N-Me-Trp(Boc)-OAll oxalate (IIa)

N-NBS-N-Me-Trp(Boc)-OAll (171 g, 314.6 mmol) and DMF (684 mL) were charged into a reaction vessel and the system was inertized and cooled to −5° C. Thiophenolate (65.0 g, 491.8 mmol) was added at −5-0° C. over 30 minutes. The reaction mixture was stirred at −5° C. for 4 hours. Tert-butyl methyl ether (1.03 L) was added, followed by water (1.28 L) at −5-0° C. (typically 30-60 minutes). The reaction mixture was then brought to 20° C. and stirred for 2 hours. The layers were separated. The organic layer was washed with water (513 mL) three times and concentrated under reduced pressure at <35° C. to 264 g. Acetone (850 mL) was added. The solution was polish filtered. The solution was cooled to 7° C. and a solution of oxalic acid dihydrate (39.2 g, 310.9 mmol) in acetone (342 mL) was added. The mixture was stirred at 7° C. for 2 hours. The precipitate was filtered off, washed with acetone (375 mL) three times and dried under reduced pressure at 35° C. for 3 hours to afford 130 g (92%) of N-Me-Trp(Boc)-OAll as a yellowish solid.

LCMS: 359.19 (M+H⁺)

### Example 3

### Preparation of tert-butyl 3-[(2S)-3-allyloxy-2-[[(2S)-6-(tert-butoxycarbonylamino)-2-[[(2S)-5-(tert-butoxycarbonylamino)-2-(9H-fluoren-9-ylmethoxycarbonylamino)pentanoyl]amino]hexanoyl]-methyl-amino]-3-oxo-propyl]indole-1-carboxylate (IVa)

H-N-Me-Trp(Boc)-OAll oxalate (IIIa oxalate, 150 g, 334.5 mmol) and tert-butyl methyl ether (1.1 kg) were charged into a reactor and water (1.95 kg) was added followed by aqueous NaOH 28% (119.5 kg) within 5 minutes (slight gas evolution). The biphasic system was stirred at room temperature for 60 minutes and the phases were separated. The organic phase was washed with water (750 g), evaporated under reduced pressure and dried under high vacuum. The residue was dissolved in n-heptane (513 g) and the solution was completely evaporated. The residue was dissolved in n-heptane (257 g) and the solution obtained kept on side (solution A). Fmoc-Orn(Boc)-Lys(Boc)-OH (IIa, 228.5 g, 334.6 mmol) and tert-butyl methyl ether (1.1 kg) were charged into a reactor. HOAt (22.75 g, 167.2 mmol) was added and the funnel was rinsed with tert-butyl methyl ether (50 g). Dimethylacetamide (155 g) was added and the addition funnel was rinsed with tert-butyl methyl ether (100 g). The mixture was cooled to 0-5° C. and diisopropylcarbodiimide (44.5 g, 352.6 mmol) was added within 10 minutes. The addition funnel was rinsed with tert-butyl methyl ether (200 g). The reaction mixture was stirred at −2-2° C. for 120 minutes. Solution A was added over 30 minutes and the addition line was rinsed with nheptane (68 g). Stirring was pursued for 40 minutes. The reaction mixture was warmed to room temperature within 180-240 minutes and stirred further for 15 hours. A solution of citric acid (103 g) and water (1.95 kg) was added within 30 minutes at T=<25° C. The dropping funnel was rinsed with water (200 g). The extraction mixture was stirred for 90 minutes, filtered through a glass filter and the reactor was rinsed with n-heptane (298 g) and the filter cake washed with a mixture of tert-butyl methyl ether (340 g) and n-heptane (157 g). The biphasic filtrate was stirred for another 10 minutes, and then the phases were separated. The organic layer was washed with a solution of NaHCO₃ (56.5 g) and water (1.25 kg), and three times with a mixture of methanol (871 g) and water (1.1 kg). The organic layer was concentrated under reduced pressure to a volume of about 900 mL (solution gets more viscous). DMSO (2.43 kg) was added and the solution was dried at <40° C. under high vacuum for 60-120 minutes. The yield was considered as 100% and the solution taken as is in the next step (ca. 12.3% w/w solution). Chiral purity (method A; % ala): LLL: 97.2%; LLD: n.d.; LDL: 2.80%; DLL: n.d.

### Example 4

### Preparation of tert-butyl 3-[(2S)-3-allyloxy-2-[[(2S)-6-(tert-butoxycarbonylamino)-2-[[(2S)-5-(tert-butoxycarbonylamino)-2-(9H-fluoren-9-ylmethoxycarbonylamino)pentanoyl]amino]hexanoyl]-methyl-amino]-3-oxo-propyl]indole-1-carboxylate (IVa)

H-N-Me-Trp(Boc)-OAll oxalate (IIIa oxalate, 80 g, 178.4 mmol) and tert-butyl methyl ether (587 g) were charged into a reactor and water (1.04 kg) was added followed by aqueous NaOH 30% (59.5 g) within 5 minutes (slight gas evolution). The biphasic system was stirred at room temperature for 60 minutes and the phases were separated. The organic phase was washed with water (400 g), evaporated under reduced pressure and dried under high vacuum. The residue was dissolved in heptane (275 g) and completely evaporated under reduced pressure to give H-N-MeTrp(Boc)OAll (IIIa, 64.6 g, 100%) as a yellow oil.

A reaction vessel was charged with Fmoc-Orn(Boc)-Lys(Boc)-OH (IIa, 20.5 g, 30.0 mmol), 2-hydroxypyridine-N-oxide (“HOPO”, 1.67 g, 15 mmol) and 1,3-dimethyl-2-imidazolidinone (“DMI”, 64.9 g). After dissolution is complete, a solution of H-N-Me-Trp(Boc)-OAll (IIIa, 11.1 g, 30.9 mmol) in tert-butyl methyl ether (40 g) was added. The reaction mixture was diluted with tert-butyl methyl ether (91 g). A solution of diisopropylcarbodiimide (“DIC”, 4.17 g, 33 mmol) in tert-butyl methyl ether (11.0 g) was added over 20 minutes. The dropping funnel was rinsed with tert-butyl methyl ether (10.0 g) and stirring was pursued at room temperature for 22 hours. n-Heptane (47.0 g) was added to the reaction mixture, followed by a solution of citric acid (11.53 g, 60 mmol) in water (220 g) within 10 minutes. The mixture was stirred for 1 hour. The precipitate was filtered off. The filter cake was washed with a mixture of tert-butyl methyl ether (57 g) and n-heptane (18 g). The phases of the filtrate were separated. The organic layer was washed with a solution of citric acid (11.5 g, 60 mmol) in water (220 g), with a solution of sodium bicarbonate (2.5 g) in water (47.5 g) and three times with a mixture of methanol (79 g) and water (100 g). The organic phase was concentrated under reduced pressure and dried under high vacuum to afford IVa (29.3 g, 95.6%) as an oil. Chiral purity (method A; % ala): LLL: 98.8%; LLD: n.d.; LDL: 1.2%; DLL: n.d.

### Example 5

### Alternative preparation of tert-butyl 3-[(2S)-3-allyloxy-2-[[(2S)-6-(tert-butoxycarbonylamino)-2-[[(2S)-5-(tert-butoxycarbonylamino)-2-(9H-fluoren-9-ylmethoxycarbonylamino)pentanoyl]amino]hexanoyl]-methyl-amino]-3-oxo-propyl]indole-1-carboxylate (IVa)

H-N-Me-Trp(Boc)-OAll oxalate (IIIa oxalate, 150 g, 334.5 mmol) and tert-butyl methyl ether (1.1 kg) were charged into a reactor and water (1.95 kg) was added followed by aqueous NaOH 28% (119.5 g) within 5 minutes (slight gas evolution). The biphasic system was stirred at room temperature for 60 minutes and the phases were separated. The organic phase was washed with water (750 g), evaporated under reduced pressure and dried under high vacuum. The residue was dissolved in heptane (513 g) and completely evaporated under reduced pressure. The residue was dissolved in heptane (356 g) (=Solution A).

A reaction vessel was charged with Fmoc-Orn(Boc)-Lys(Boc)-OH (IIa, 228.0 g, 334.5 mmol), 1-hydroxy-7-azabenzotriazole (22.8 g, 167.1 mmol), dimethylacetamide (155 g), and tert-butyl-methyl ether (1.25 kg). To the suspension obtained cooled to 0° C. were added diisopropylcarbodiimide (44.5 g, 352.6 mmol) and and tert-butyl methyl ether (200 g). The reaction mixture was stirred at 0° C. for 2 hours. Solution A was added within 30 minutes. The addition funnel was rinsed with nheptane (68 g). The reaction mixture was stirred for 40 minutes, then warmed to 22° C. within 3.5 hours, and stirred 16 hours at this temperature. A solution of citric acid (103 g, 536 mmol) in water (2.15 kg) was added within 30 minutes. The reaction mixture was stirred for 90 minutes, diluted with heptane (297 g) and filtered. The precipitate was washed with a mixture of tert-butyl methyl ether (340 g) and heptane (157 g). The phases of the filtrate were separated. The organic layer was washed with a solution of sodium bicarbonate (56.5 g) in water (1.25 kg) then 3 times with a mixture of methanol (871 g) and water (1.1 kg). The organic phase was concentrated under reduced pressure to a volume of ca. 900 mL, diluted with DMSO (2.43 kg) and dried under high vacuum to afford IVa as a 12.3% wt solution in DMSO. Chiral purity (method A; % a a): LLL: 97.2%; LLD: n.d.; LDL: 2.8%; DLL: n.d.

### Comparative Example 6

Table 1 shows a screening of reaction conditions for the coupling of amine IIIa with carboxylic acid IIa (see also Examples 4 and 5 above).

TABLE 1

| | | | | | Final IPC, | ratio |
|---|---|---|---|---|---|---|
| Exp | Reagent (1.5 | Base | Solvent | Reaction | area % | LLL/LDL |
| no | eq) | (3 eq) | (20 rel vol) | condition | Product | epimer |
| 1 | HATU | DIPEA | DCM/DMF | 0° C., 20 rel | 65.2 | 56.9/43.1 |
| | | | 3/1 | vol | | |
| 2 | TBTU | DIPEA | DCM/DMF | 0° C., 20 rel | 45.3 | 52.7/47.3 |
| | | | 3/1 | vol | | |
| 3 | T3P | NMM | EtOAc/DCM | Rt, 10 rel | 93.0 | 56.3/43.7 |
| | | | | vol | | |
| 4 | T3P | NMM | EtOAc/DCM | 0° C., 20 rel | 75.9 | 59.5/40.5 |
| | | | | vol | | |
| 5 | T3P | Pyridine | EtOAc/DCM | 0° C., 20 rel | 69.9 | 61.0/39.0 |
| | | | | vol | | |
| 6 | HATU | DIPEA | DCM/DMF | 0° C. 1 h-rt, | 64.8 | 57.1/42.9 |
| | | | 3/1 | 20 rel vol | | |
| 7 | TBTU | DIPEA | DCM/DMF | 0° C. 1 h-rt, | 41.8 | 52.3/43.7 |
| | | | 3/1 | 20 rel vol | | |
| 8 | EDCI/HOBt | — | DCM | 0° C. 1 h-rt, | 59.2 | 57.1/42.9 |
| | | | | 20 rel vol | | |
| 9 | EDCI/HOAt | — | DCM | 0° C. 1 h-rt, | 90.3 | 87.0/13.0 |
| | | | | 20 rel vol | | |
| 10 | DIC/HOBt | — | DCM | 0° C. 1 h-rt, | 72.5 | 83.6/16.4 |
| | | | | 20 rel vol | | |
| 11 | DIC/HOAt | — | DCM | 0° C. 1 h-rt, | 88.2 | 97.7/2.3 |
| | | | | 20 rel vol | | |
| 12 | EDCI/Oxyma | | DCM | 0° C. 10 | 40.4 | 71.3/28.7 |
| | | | | min-rt, 20 | | |
| | | | | rel vol | | |
| 13 | PyBroP | DIPEA | DCM/DMF | 0° C. 1 h-rt, | Very little | |
| | | | 3/1 | 20 rel vol | product | |
| | | | | | formed | |
| 14 | TOTT | DIPEA | DCM/DMF | 0° C. 1 h-rt, | 76.9 | 69.9/30.1 |
| | | | 3/1 | 20 rel vol | | |
| 15 | DMTMM | NMM | DCM/DMF | 0° C. 1 h-rt, | 54.9 | 63.4/36.6 |
| | | | 3/1 | 20 rel vol | | |
| 16 | BOP-Cl | NMM | DCM/DMF | 0° C. 1 h-rt, | 29.5 | 54.3/45.7 |
| | | | 3/1 | 20 rel vol | | |
| 17 | DIC/HODhat | | DCM | 0° C. 1 h-rt, | 77.7 | 98.9/1.1 |
| | | | | 20 rel vol | | |
| 18 | DIC/HOSu | — | DCM | 0° C. 1 h-rt, | Very little | |
| | | | | 20 rel vol | product | |
| | | | | | formed | |
| 19 | DIC/Oxyma | — | DCM | 0° C. 1 h-rt, | 77.1 | 78.2/21.8 |
| | | | | 20 rel vol | | |
| 20 | DIC/2- | — | DCM/DMF | 0° C. 1 h-rt, | 51.9 | 57/43 |
| | Hydroxypyridine | | | 20 rel vol | | |
| 21 | DIC/HOBt | Pyridine | DCM | 0° C. 1 h-rt, | 65.6 | 87.3/16.6 |
| | | | | 20 rel vol | | |
| 22 | DIC/HOBt | Pyridine | DMF | 0° C. | 29.7 | 48.5/51.5 |
| | | | | 10 min-rt, | | (interfering |
| | | | | 20 rel vol | | peeks) |
| 23 | COMU | DIPEA | DMF | 0° C. | 80.0 | 58.3/41.7 |
| | | | | 10 min-rt, | | |
| | | | | 20 rel vol | | |
| 24 | DIC/6-Cl- | — | DCM | 0° C. 1 h-rt, | 72.9 | 91.2/8.8 |
| | HOBt | | | 20 rel vol | | |

### Example 7

### tert-butyl 3-[(2S)-3-allyloxy-2-[[(2S)-2-[[(2S)-2-amino-5-(tert-butoxycarbonylamino)pentanoyl]amino]-6-(tert-butoxycarbonylamino)hexanoyl]-methyl-amino]-3-oxo-propyl]indole-1-carboxylate (Va)

The solution of tert-butyl 3-[(2S)-3-allyloxy-2-[[(2S)-6-(tert-butoxycarbonylamino)-2-[[(2 S)-5-(tert-butoxycarbonylamino)-2-(9H-fluoren-9-ylmethoxycarbonylamino)pentanoyl]amino]hexanoyl]-methyl-amino]-3-oxo-propyl]indole-1-carboxylate in DMSO from Example 5 (IVa, 2.43 kg, 334.2 mmol) was charged into a reactor. N-Acetyl-cysteine (71.0 g, 435.1 mmol, 1.3 eq.) was added and the addition funnel rinsed with DMSO (100 g). tert-Amylamine (75.8 g, 869.0 mmol, 2.6 eq.) was added (slightly exothermic) and the addition funnel was rinsed with DMSO (85 g). The reaction mixture was stirred at 19-21° C. for 90 minutes and a solution of acetic acid (52.18 g, 869.0 mmol, 2.6 eq.) in water (680 g) was added dropwise keeping T=<22° C. (exothermic). The quenched reaction mixture was added to a solution of KHCO₃ (87.0 g, 869.0 mmol, 2.6 eq.) in water (2.91 kg) and tert-butyl methyl ether (2.0 kg) keeping temperature=<25° C. After 30 minutes stirring, the phases were separated. The organic layer was washed with a solution of K₂CO₃ (92.4 g, 668.5 mmol, 2.0 eq.) in water (2.84 kg) and with water (1.7 kg). The organic phase was concentrated under reduced pressure to a volume of 870 mL. Toluene (865 g) was added and the solution was concentrated to 870 mL and diluted with toluene (216.2 g). Yield based on assay of solution (31.86% w/w) was 93.6%. Chiral purity (method B; % a a): LLL: 97.42%; LLD: n.d.; LDL: 2.58%; DLL: n.d.

### Example 8

### Alternative preparation of tert-butyl 3-[(2S)-3-allyloxy-2-[[(2S)-2-[[(2S)-2-amino-5-(tert-butoxycarbonylamino)pentanoyl]amino]-6-(tert-butoxycarbonylamino)hexanoyl]-methyl-amino]-3-oxo-propyl]indole-1-carboxylate (Va)

A reaction vessel was charged with a solution of Fmoc-Orn(Boc)-Lys(Boc)-N(Me)-Trp(Boc)-OAll (IVa, 177.2 g, 173.2 mmol) and 1093 mL DMSO at room temperature. N-Acetyl-cysteine (36.7 g, 1.3 eq.) was added and the funnel rinsed with DMSO (100 mL). The reaction mixture was stirred until the solids were completely dissolved. A solution of tert-butylamine (32.9 g, 2.6 eq.) in DMSO (47 mL) was added over 30-60 minutes keeping the temperature at 18-22° C. The reaction mixture was stirred at 18-22° C. for 90 minutes. tert-Butyl methyl ether (1400 mL) was added. The mixture was cooled to 10-15° C. A solution of acetic acid (27.1 g, 2.6 eq.) in water (354 mL) was added dropwise keeping the temperature below 20° C. The quenched reaction mixture was stirred for 30 minutes and a solution of potassium bicarbonate (38.0 g, 2.2 eq.) in water (1220 mL) was added keeping the temperature below 20° C. After 30 minutes stirring, the phases were separated. The organic layer was washed with a solution of potassium carbonate (47.6 g, 2.0 eq.) in water (1452 mL), then with water (708 mL). The organic layer was dried on MgSO₄ (104 g). The solid was filtered off and washed with tert-butyl methyl ether (138 mL). The filtrate was concentrated to ca 2.0 volumes. The residue was diluted with tert-butyl methyl ether (416 mL) and n-heptane (416 mL) was added (=solution A). The solution was cooled to 0-5° C. A solution of phosphoric acid (85%, 8.45 g, 0.43 eq.) in tert-butyl methyl ether (138.5 ml) was prepared at 5-10° C. and cooled to 0-5° C. The phosphoric acid solution was added to the solution A keeping the temperature at 0-5° C. (ca. 1-1.5 h). The suspension was stirred at 0-5° C. for two more hours. Cold n-heptane (3050 mL) were slowly added and stirring was pursued at 0-5° C. for 1 hour. The precipitate was filtered off, washed with cold n-heptane (277 mL) and dried under reduced pressure at 25° C. to afford Orn(Boc)-Lys(Boc)-N(Me)-Trp(Boc)-OAll hemiphosphate (Va) as a yellowish solid (112.0 g, 76.2% over two steps).

### Example 9

### tert-butyl 3-[(2S)-3-allyloxy-2-[[(2S)-2-[[(2S)-2-[[2-[3-bromo-2-[(9H-fluoren-9-ylmethoxycarbonylamino)methyl]phenyl]sulfanyl-3-pyridyl]methylamino]-5-(tert-butoxycarbonylamino)pentanoyl]amino]-6-(tert-butoxycarbonylamino)hexanoyl]-methyl-amino]-3-oxo-propyl]indole-1-carboxylate (VIIa)

3-[(2S)-3-allyloxy-2-[[(2S)-2-[[(2S)-2-amino-5-(tert-butoxycarbonylamino)pentanoyl]amino]-6-(tert-butoxycarbonylamino)hexanoyl]-methyl-amino]-3-keto-propyl]indole-1-carboxylic acid tert-butyl ester (Va, 209 g, 260.9 mmol, 1.0 eq.) dissolved in toluene (447.3 g, 656.3 g solution) was charged into a reaction vessel and the solution was diluted with toluene (1.11 kg, 1.28 L.) to afford a yellow clear solution. N-[2-bromo-6-[(3-formyl-2-pyridyl)thio]benzyl]carbamic acid 9H-fluoren-9-ylmethyl ester (142.32 g, 260.9 mmol, 1 eq.) was added at room temperature and the addition funnel was rinsed with toluene (144.52 mL). This afforded a light yellow suspension. Acetic acid (34.47 g, 574.05 mmol, 2.2 eq.) was added at room temperature and the funnel was rinsed with toluene (24.12 mL). At Ti=45-50° C. and 100-150 mbar, 836 mL toluene was distilled off over about 2 hours (azeotropic water removal). Around 45° C. the suspension turns to a solution. Acetic acid (15.67 g, 260.9 mmol, 1.0 eq.) was added at room temperature, followed by sodium triacetoxyborohydride (110.6 g, 521.86 mmol, 2.0 eq.). The addition funnel was rinsed with toluene (836 mL). The reaction mixture was stirred at 40° C. for 3 hours. Subsequently, the reaction mixture was added to a solution of sodium bicarbonate (131.52 g, 1565.58 mmol, 6 eq) and water (1.92 L) over 15 minutes. During the addition, gas evolution and foaming was observed. The extraction mixture was heated to 50° C. and stirred for 40 minutes (gas evolution and foaming; pH=7), then kept at room temperature overnight without stirring. The layers were separated. The organic layer was washed with water (1.28 L). The organic phase was concentrated under reduced pressure and dried under high vacuum at 40° C. for 4 hours to afford the title compound VIIa (371 g) as a yellow foam. Yield considered as 100% for the next step.

### Example 10

### (2S)-2-[[(2S)-2-[[(2S)-2-[[2-[2-(aminomethyl)-3-bromo-phenyl]sulfanyl-3-pyridyl]methylamino]-5-(tert-butoxycarbonylamino)pentanoyl]amino]-6-(tert-butoxycarbonylamino)hexanoyl]-methyl-amino]-3-(1-tert-butoxycarbonylindol-3-yl)propanoic acid (IXa)

3-[(2S)-3-allyloxy-2-[[(2S)-2-[[(2S)-2-[[2-[[3-bromo-2-[(9H-fluoren-9-ylmethoxycarbonylamino)methyl]phenyl]thio]-3-pyridyl]methylamino]-5-(tert-butoxycarbonylamino)pentanoyl]amino]-6-(tert-butoxycarbonylamino)hexanoyl]-methyl-amino]-3-keto-propyl]indole-1-carboxylic acid tert-butyl ester (VIIa, 347.15 g, 0.261 mol,) in acetonitrile (1.1 L) was charged into reaction vessel. Diethylamine (95.42 g, 1.3 mol, 5.0 eq.) was added at room temperature and the addition funnel was rinsed with acetonitrile (28 mL). Palladium tetrakis(triphenylphosphine) (1.51 g, 0.001 mol, 0.005 eq.) was added at room temperature (slightly exothermic) and the addition funnel was rinsed with acetonitrile (68 mL). The reaction mixture was stirred at room temperature for 45 minutes. N-Acetyl-cysteine (51.1 g, 0.313 mol, 1.2 eq.) was added, the addition funnel was rinsed with acetonitrile (435.1 mL), and the reaction mixture was stirred at 50° C. for 2.5 hours. The reaction mixture was further stirred at room temperature overnight. Dicyclohexylamine (61.51 g, 0.339 mol, 1.3 eq.) was added and the addition funnel rinsed with acetonitrile (28 mL). The reaction mixture was stirred 1 hour at room temperature (after ca 10 minutes the solution turns into a suspension). At Ti max 35° C./180-160 mbar and constant volume acetonitrile (2.26 L), diethylamine was removed by azeotropic distillation The reaction mixture was filtered off, the reactor and filter cake were washed with acetonitrile (1.1 L). The filtrate was evaporated under reduced pressure at 40° C. and the residue was dried under high vacuum for 2 hours.

### Example 11

### Alternative preparation of (2S)-2-[[(2S)-2-[[(2S)-2-[[2-[2-(aminomethyl)-3-bromo-phenyl]sulfanyl-3-pyridyl]methylamino]-5-(tert-butoxycarbonylamino)pentanoyl]amino]-6-(tert-butoxycarbonylamino)hexanoyl]-methyl-amino]-3-(1-tert-butoxycarbonylindol-3-yl)propanoic acid (IXa)

Crude tert-butyl 3-[(2S)-3-allyloxy-2-[[(2S)-2-[[(2S)-2-[[2-[3-bromo-2-[(9H-fluoren-9-ylmethoxycarbonylamino)methyl]phenyl]sulfanyl-3-pyridyl]methylamino]-5-(tert-butoxycarbonylamino)pentanoyl]amino]-6-(tert-butoxycarbonylamino)hexanoyl]-methyl-amino]-3-oxo-propyl]indole-1-carboxylate (VIIa, 43.6 g, 31.2 mmol) was dissolved in acetonitrile (211.6 g). The solution was concentrated under reduced pressure to a volume of 100-110 mL and diluted with acetonitrile (218.0 g). Diethylamine (22.8 g, 10.0 eq.) was added and the addition funnel was rinsed with acetonitrile (4.2 g). Palladium tetrakis(triphenylphosphine) (180.4 mg, 0.005 eq.) was added and the funnel was rinsed with acetonitrile (2.1 g). The reaction mixture was stirred at room temperature for 1.5 hour. N-Acetyl-cysteine (6.1 g, 1.2 eq.) was added. The funnel was rinsed with acetonitrile (4.2 g) and the reaction mixture was stirred at room temperature for 3 hours. Dicyclohexylamine (7.4 g, 1.3 eq.) was added and the transfer line was rinsed with acetonitrile (4.2 g). The reaction mixture was distilled at 45° C. under reduced pressure at constant volume with the addition of acetonitrile (218.0 g). The suspension formed is cooled to room temperature and stirred for 1 hours at room temperature, cooled to 0° C. over 3 hours and stirred at 0° C. for 10 hours. The precipitate was filtered off, washed with cold acetonitrile (54.5 g) and the filtrate evaporated under reduced pressure and dried under high vacuum. The residue was used as is and assuming 100% yield in the next step.

### Example 12

### tert-butyl 3-[[(11S,14S,17S)-22-bromo-14-[4-(tert-butoxycarbonylamino)butyl]-11-[3-(tert-butoxycarbonylamino)propyl]-16-methyl-12,15,18-trioxo-2-thia-4,10,13,16,19-pentazatricyclo[19.4.0.03,8]pentacosa-1(25),3,5,7,21,23-hexaen-17-yl]methyl]indole-1-carboxylate (Ia)

1-Hydroxybenzotriazole hydrate (79.92 g, 0.522 mol, 4.0 eq.), 1-(3-dimethylaminopropyl)-3-ethylcarbodiimide hydrochloride (100.05 g, 0.522 mol, 4.0 eq.) and dichloromethane (1.07 L) were charged into a reactor at room temperature (endothermic Ti 13° C.). The suspension formed slowly a solution. A solution of (2S)-2-[[(2S)-2-[[(2S)-2-[[2-[[2-(aminomethyl)-3-bromo-phenyl]thio]-3-pyridyl]methylamino]-5-(tert-butoxycarbonylamino)pentanoyl]amino]-6-(tert-butoxycarbonylamino)hexanoyl]-methyl-amino]-3-(1-tert-butoxycarbonylindol-3-yl)propionic acid (IXa, 139.36 g, 0.130 mol,) in dichlormethane (695 mL) was added at room temperature within 4 hours. The addition funnel was rinsed with dichloromethane (92.8 mL). The reaction mixture was stirred at room temperature for another 30 minutes. A solution of sodium bicarbonate (48.95 g, 0.583 mol, 4.47 eq.) in water (670.0 mL) was added within 30 minutes (gas evolution) und stirring was pursued for 30 to 40 minutes. The phases were separated. The organic layer was washed twice with a solution of sodium bicarbonate (48.95 g, 0.583 mol, 4.47 eq.) in water (670.0 mL), then with water (670.0 mL). The organic phase was concentrated at 25-35° C./700-600 mbar to a volume of 479 mL. Crystals started to precipitate during the distillation. At constant volume and 48-52° C./600-300 mbar the solvent was swapped to ethyl acetate (1.1 L). The fine suspension obtained was cooled to room temperature within 30 minutes and stirred for another hour at room temperature. n-Heptane (349.15 mL) was added within 60 minutes and the suspension stirred overnight at room temperature. The precipitate was filtered off and the filter cake washed with a mixture of ethyl acetate (157.3 mL) and n-heptane (157.3 mL). The powder was dried at 50° C. under high vacuum to afford the title compound Ia (92.8 g, 67.7%) as a beige powder. Chiral purity (method C; % ala): 0.02% DLL; 99.18% LLL; 0.52% LDL; 0.29% LLD.

This product was further purified: compound Ia obtained (90.63 g) was dissolved at room temperature in dichloromethane (406 mL) and filtered on a charcoal pad. The filter was washed with dichloromethane (362 mL). The filtrate was concentrated under reduced pressure to a volume of 447 mL. Ethyl acetate (555 mL) was added and the solvent was exchanged at constant volume (1002 mL) under reduced pressure and at internal temperature of 32-65° C. with ethyl acetate (1590 mL). Ethyl acetate (394 mL) was distilled off under reduced pressure at 60-65° C. The orange brown suspension was cooled slowly to room temperature (0.15K/min) and aged for 16 hours. nHeptane (203 mL) were added within 45 minutes and the suspension was stirred for two hours. The precipitate was filtered off, washed with a mixture of ethyl acetate (270 mL) and nheptane (89 mL) (in two portions, slow filtration) and dried under high vacuum at 60° C. to afford Ia (62.26 g, 61%) as a white powder. Chiral purity (method C; % ala): 100%

### Example 13

### tert-butyl 3-[(2S)-3-allyloxy-2-[[(2S)-2-[[(2S)-2-amino-5-(tert-butoxycarbonylamino)pentanoyl]amino]-6-(tert-butoxycarbonylamino)hexanoyl]-methyl-amino]-3-oxo-propyl]indole-1-carboxylate (Va)

A solution of tert-butyl 3-[(2S)-3-allyloxy-2-[[(2S)-6-(tert-butoxycarbonylamino)-2-[[(2S)-5-(tert-butoxycarbonylamino)-2-(9H-fluoren-9-ylmethoxycarbonylamino)pentanoyl]amino]hexanoyl]-methyl-amino]-3-oxo-propyl]indole-1-carboxylate (IVa, 19.5 g, 19.1 mmol, 1 eq.) in MeCN (210 mL) was charged into a reactor. tert-Amylamine (5.32 g, 61 mmol, 3.2 eq.) and a suspension of N-Acetyl-cysteine (4.04 g, 24.8 mmol, 1.3 eq.) and seed crystals (of the DBF-N-acetylcysteine adduct tAmylamine salt) in MeCN (15 mL) were charged. The addition funnel was washed into the reactor with MeCN (15 mL). The reaction mixture was stirred at RT for 20 h (IPC: mostly deprotected) then 7 h at 50° C. (complete deprotection and mostly N-Acetyl-cysteine-DBF adduct). The reaction mixture was cooled to RT and filtered to remove the majority of the N-Acetyl-cysteine-DBF adduct. The filter cake was washed with MeCN (100 mL). The filtrate was concentrated under reduced pressure (50° C. 300-60 mbar). The residue was redissolved Me-THF (250 mL) and was washed sequentially twice with a solution of K₂CO₃ (25 g) in water (225 g) and a solution of NaCl (28 g) in water (200 mL). The solution was concentrated to dryness under reduced pressure (50° C., 350-15 mbar) to give the product (14.8 g as an oil) in 97% crude yield (85a % by LC at 230 nm)

1. A process for manufacturing a compound of formula (I), or a salt thereof, 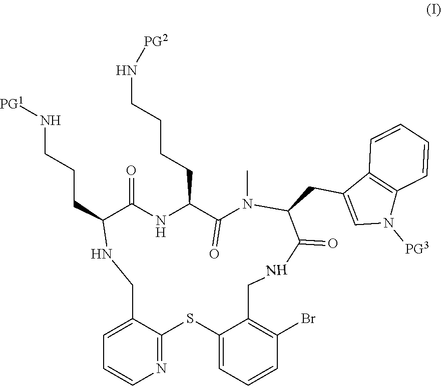
comprising:
(a) reacting a carboxylic acid of formula (II) 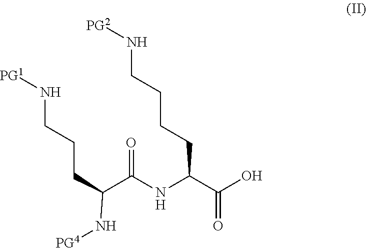
with a secondary amine of formula (III) 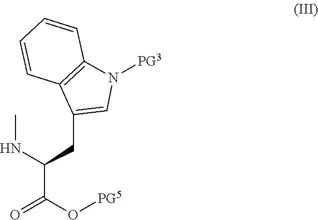
using reagents selected from:
(i) HOAt and DIC;
(ii) HODhat and DIC;
(iii) HOPO and DIC;
(iv) HOPO and DCC; and
(v) HOPO and EDC;
to form a compound of formula (IV) 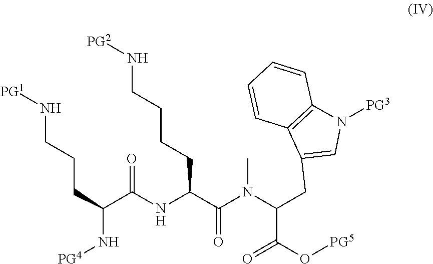
wherein PG¹, PG², PG³ and PG⁴ are amino protective groups independently selected from BOC, Adoc, Moz, and Fmoc, and PG⁵ is a carboxylic acid protective group selected from allyl and 9-fluorenylmethyl.

2. The process according to claim 1, wherein the process is performed in a solvent selected from:
(i) a mixture of tert-butyl methyl ether, n-heptane and dimethylacetamide;
(ii) isopropylacetate with or without 1,3-dimethyl-2-imidazolidinone;
(iii) tert-butyl methyl ether with or without 1,3-dimethyl-2-imidazolidinone;
(iv) dichloromethane with or without 1,3-dimethyl-2-imidazolidinone;
(v) THF with or without 1,3-dimethyl-2-imidazolidinone;
(vi) 2-methyl-THF with or without 1,3-dimethyl-2-imidazolidinone;
(vii) toluene with or without 1,3-dimethyl-2-imidazolidinone; and
(viii) acetonitrile.

3. The process according to claim 1, further comprising:
(b1) reacting said compound of formula (IV), wherein PG⁴ is Fmoc, with N-acetylcysteine and tAmNH₂ or tBuNH₂, to form a compound of formula (V): 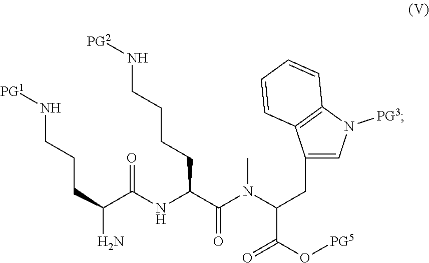
wherein PG¹, PG², and PG³ are amino protective groups independently selected from BOC, Adoc, Moz, and Fmoc, and PG⁵ is a carboxylic acid protective group selected from allyl and 9-fluorenylmethyl; and
(b2) washing the reaction mixture obtained in (b1) with a basic aqueous solution.

4. The process according to claim 3, further comprising:
(c) reacting said compound of formula (V), with a compound of formula (VI): 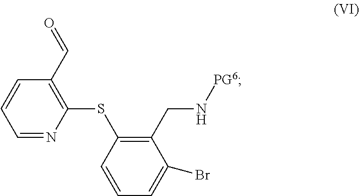
wherein PG⁶ is an amino protective group selected from BOC, Adoc, Moz, and Fmoc; in the presence of (i) a reducing agent selected from NaBH₃CN and NaBH(OAc)₃; and (ii) a carboxylic acid selected from acetic acid and propionic acid; to form a compound of formula (VII): 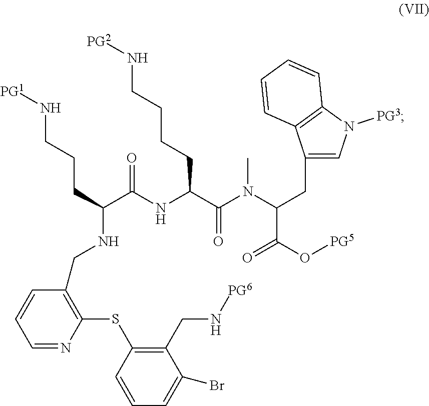
wherein PG¹, PG², PG³ and PG⁶ are amino protective groups independently selected from BOC, Adoc, Moz, and Fmoc, and PG⁵ is a carboxylic acid protective group selected from allyl and 9-fluorenylmethyl.

5. The process according to claim 4, further comprising:
(d) reacting said compound of formula (VII), wherein PG⁵ is an allyl group, with a transition metal catalyst in the presence of a secondary amine, to form a compound of formula (IX): 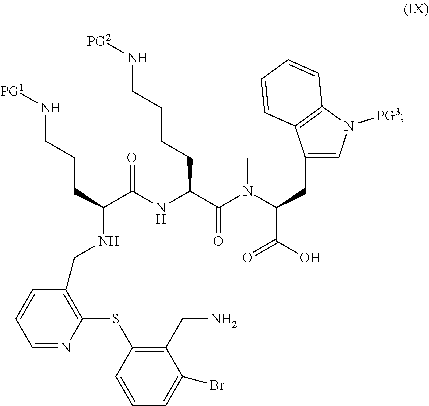
wherein PG¹, PG² and PG³ are amino protective groups independently selected from BOC, Adoc, Moz, and Fmoc.

6. The process according to claim 5, wherein the secondary amine is Et₂NH and the transition metal catalyst is (PPh₃)₄Pd.

7. The process according to claim 5, wherein step (d) is performed in acetonitrile and further comprises working up the reaction mixture obtained from step (d) by:
(d2) adding N-acetylcysteine to said reaction mixture obtained from step (d);
(d3) adding Cy₂NH to the reaction mixture obtained from step (d2);
(d4) distilling off the secondary amine from step (d); and
(d5) filtering the reaction mixture obtained from step (d4).

8. The process according to claim 5, further comprising:
(e) reacting said compound of formula (IX) with:
(i) a mixture of HOBt and EDCI;
(ii) a mixture of DIC and oxyma;
(iii) COMU;
(iv) 2,4,6-trichloro-1,3,5-triazine (TCT); or
(v) 4-(4,6-dimethoxy-1,3,5-triazin-2-yl)-4-methyl-morpholinium chloride (DMTMM);
to form a compound of formula (I).

9. A compound of formula (II), 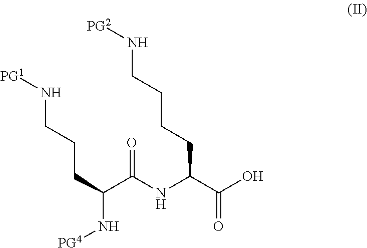
or a salt thereof, wherein PG¹, PG² and PG⁴ are amino protective groups independently selected from BOC, Adoc, Moz, and Fmoc.

10. A compound of formula (III), 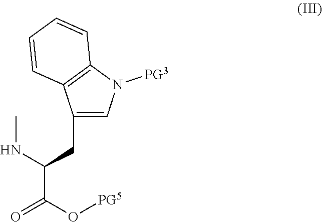
or a salt thereof, wherein PG³ is an amino protective group selected from BOC, Adoc, Moz, and Fmoc, and PG⁵ is a carboxylic acid protective group selected from allyl and 9-fluorenylmethyl.

11. A compound of formula (IV), 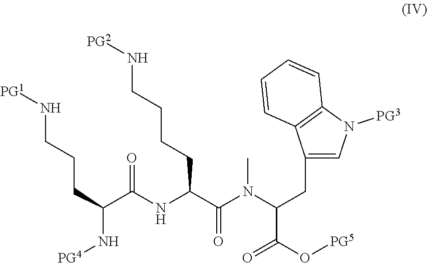
or a salt thereof, wherein PG¹, PG², PG³ and PG⁴ are amino protective groups independently selected from BOC, Adoc, Moz, and Fmoc, and PG⁵ is a carboxylic acid protective group selected from allyl and 9-fluorenylmethyl.

12. A compound of formula (V), 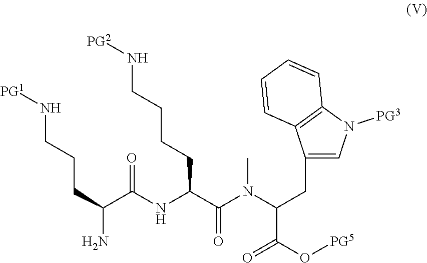
or a salt thereof, wherein PG¹, PG² and PG³ are amino protective groups independently selected from BOC, Adoc, Moz, and Fmoc, and PG⁵ is a carboxylic acid protective group selected from allyl and 9-fluorenylmethyl.

13. A compound of formula (VII), 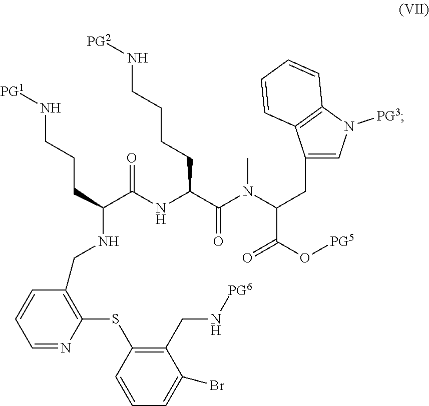
or a salt thereof, wherein PG¹, PG², PG³ and PG⁶ are amino protective groups independently selected from BOC, Adoc, Moz, and Fmoc, and PG⁵ is a carboxylic acid protective group selected from allyl and 9-fluorenylmethyl.

14. A compound of formula (VIII), 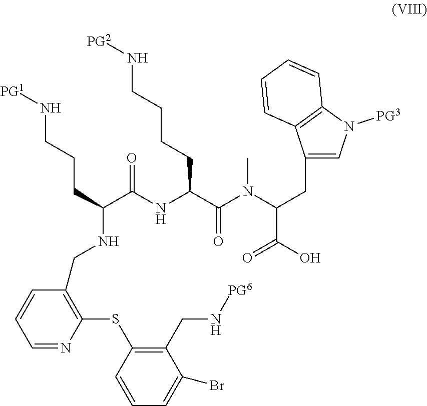
or a salt thereof, wherein PG¹, PG² and PG³ and PG⁶ are amino protective groups independently selected from BOC, Adoc, Moz, and Fmoc.

15. A compound of formula (IX), 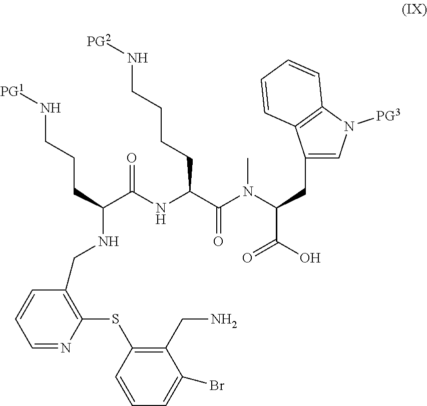
or a salt thereof, wherein PG¹, PG² and PG³ are amino protective groups independently selected from BOC, Adoc, Moz, and Fmoc.

16. The process according to claim 1, wherein PG¹, PG² and PG³ are each BOC; PG⁴ is Fmoc; and PG⁵ is allyl.

17. The process according to claim 3, wherein PG¹, PG² and PG³ are each BOC; and PG⁵ is allyl.

18. The process according to claim 4, wherein PG¹, PG² and PG³ are each BOC; PG⁵ is allyl; and PG⁶ is Fmoc.

19. The process according to claim 6, wherein PG¹, PG² and PG³ are each BOC; and PG⁶ is Fmoc.

20. The process according to claim 7, wherein PG¹, PG² and PG³ are each BOC.

21. A compound, which is N-NBS-N-Me-Trp-OAll 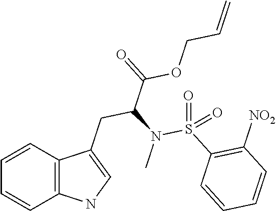
or a salt thereof.

22. A compound, which is N-NBS-N-Me-Trp(Boc)-OAll 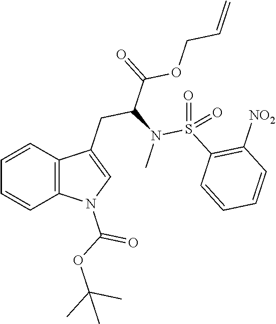
or a salt thereof.

23. A process for manufacturing a compound of formula (Ia), or a salt thereof.

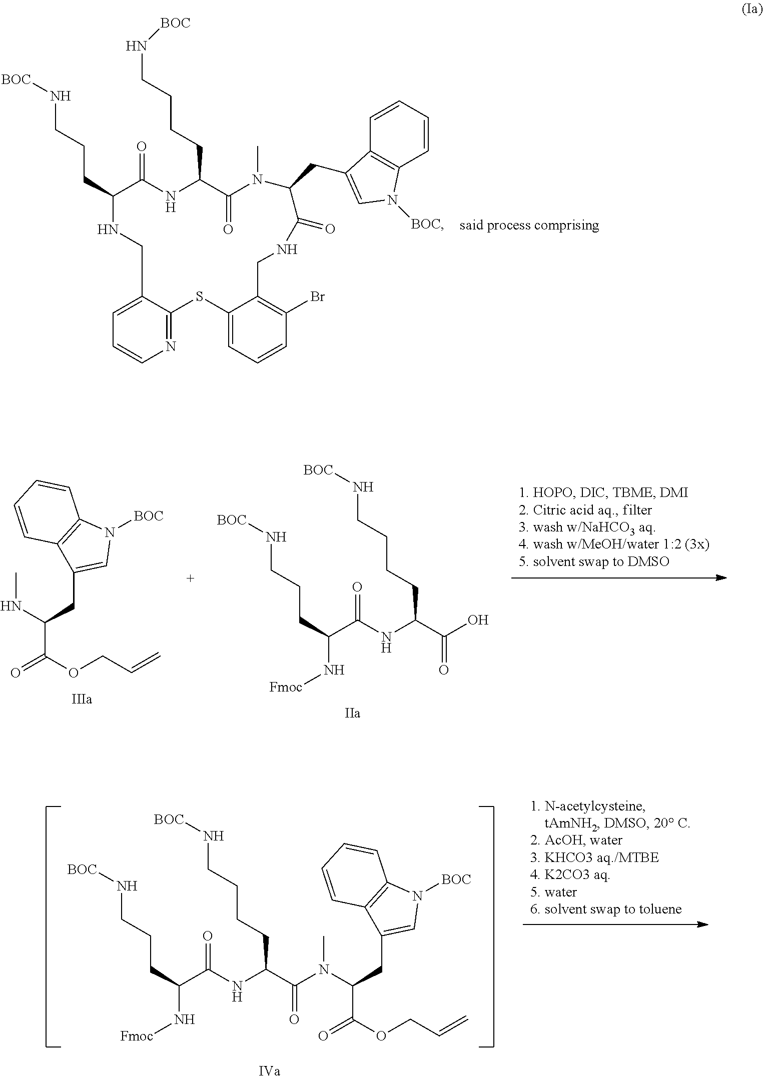

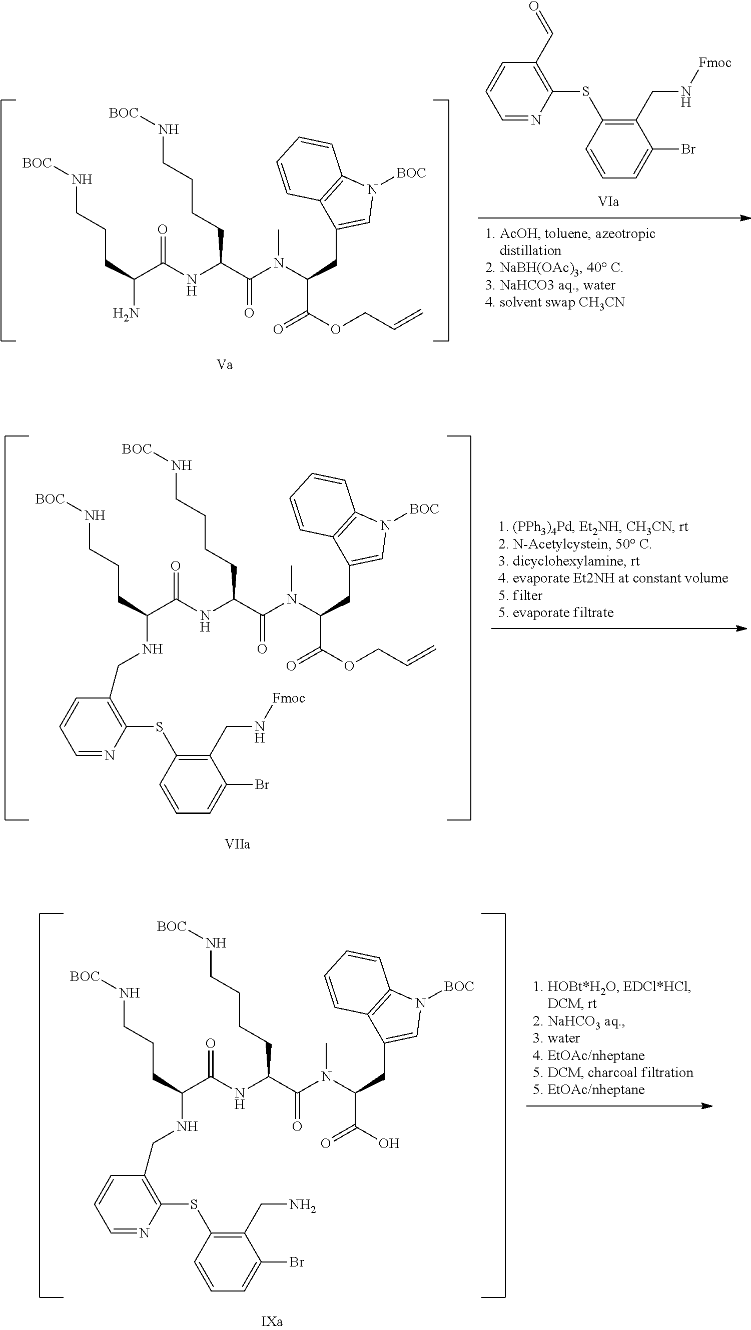

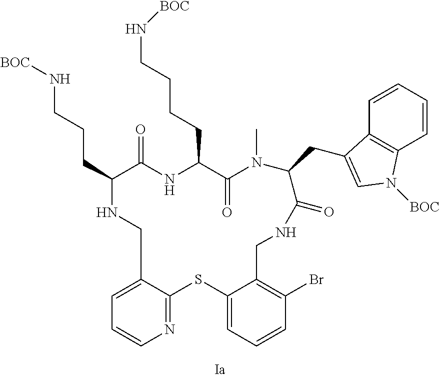 [SMILES: CN1C(=O)[C@H](CCCCNC(=O)OC(C)(C)C)NC(=O)[C@H](CCCNC(=O)OC(C)(C)C)NCc2cccnc2Sc2cccc(Br)c2CNC(=O)[C@@H]1Cc1cn(C(=O)OC(C)(C)C)c2ccccc12]

24. A compound of formula (I), or a salt thereof, 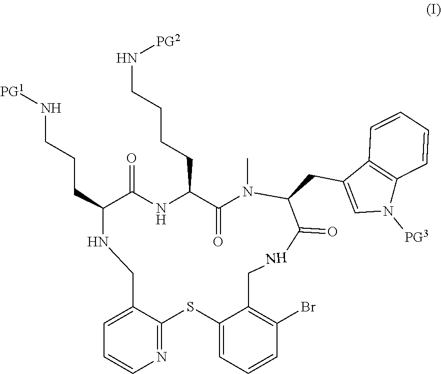
wherein PG¹, PG² and PG³ are amino protective groups.

25. A process for manufacturing a compound of formula (1), or a salt thereof, 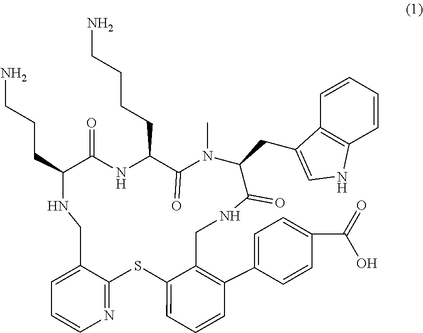 [SMILES: CN1C(=O)[C@H](CCCCN)NC(=O)[C@H](CCCN)NCc2cccnc2Sc2cccc(-c3ccc(C(=O)O)cc3)c2CNC(=O)[C@@H]1Cc1c[nH]c2ccccc12]
comprising a process according to claim 1.

26. (canceled)

27. A method for removing an Fmoc protective group from a compound comprising an Fmoc-protected amine, comprising:
(a) reacting said compound comprising an Fmoc-protected amine with a base;
(b) adding N-acetylcysteine to the reaction mixture obtained from step (a);
(c) provided the base in step (a) was not Cy₂NH or t-amylamine, adding a base selected from Cy₂NH and t-amylamine to the reaction mixture obtained from step (b);
(d) optionally distilling off the base from step (a); and
(e) filtering the reaction mixture obtained from step (b), (c) or (d).

28. The method of claim 27, wherein the method is performed in acetonitrile as a solvent.

29. The method according to claim 27, wherein the base used in step (a) is diethylamine.

30. The method according to claim 27, wherein ≥10 equivalents of base are used relative to said compound comprising an Fmoc-protected amine in step (a).

31. The method according to claim 30, wherein steps (a)-(c) are performed at room temperature.

32. The method according to claim 27, wherein the base used in step (c) is Cy₂NH.

33. The method according to claim 27, step (d) comprises distilling off the base from step (a).

34. The method according to claim 27, wherein the reaction mixture obtained in step (b) is heated to 30° C. to reflux.

35. The method of claim 34, wherein the reaction mixture obtained in step (b) is heated to 30° C. to 70° C.

36. The method of claim 35, wherein the reaction mixture obtained in step (b) is heated to 40° C. to 60° C.

37. The method of claim 36, wherein the reaction mixture obtained in step (b) is heated to 50° C.

38. The method according to claim 27, wherein said compound comprising an Fmoc-protected amine further comprises at least one carboxylic acid moiety.

39. (canceled)

40. The compound according to claim 11, wherein PG¹, PG² and PG³ are each BOC; PG⁴ is Fmoc; and PG⁵ is allyl.

41. The compound according to claim 12, wherein PG¹, PG² and PG³ are each BOC; and PG⁵ is allyl.

42. The compound according to claim 13, wherein PG¹, PG² and PG³ are each BOC; PG⁵ is allyl; and PG⁶ is Fmoc.

43. The compound according to claim 14, wherein PG¹, PG² and PG³ are each BOC; and PG⁶ is Fmoc.

44. The compound according to claim 15, wherein PG¹, PG² and PG³ are each BOC.
